(19) United States
(12) Patent Application Publication
(10) Pub. No.: US 2005/0049582 A1
(43) Pub. Date: Mar. 3, 2005

(54) Method and apparatus for fractional photo therapy of skin

(76) Inventors: Leonard C. DeBenedictis, Palo Alto, CA (US); G. Scott Herron, La Honda, CA (US); Robert Kehl Sink, Mountain View, CA (US); David Eimerl, Livermore, CA (US); Vladimir Lemberg, Redwood City, CA (US); George Voevodkin, Tarrytown, NY (US); Michael Black, Foster City, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 10/888,356

(22) Filed: Jul. 9, 2004

Related U.S. Application Data
(63) Continuation-in-part of application No. 10/367,582, filed on Feb. 14, 2003.
Application No. 10/367,582 is a continuation-in-part of application No. 10/279,093, filed on Oct. 22, 2002, now abandoned.
Application No. 10/367,582 is a continuation-in-part of application No. 10/278,582, filed on Oct. 23, 2002.
Application No. 10/278,582 is a continuation-in-part of application No. 10/017,287, filed on Dec. 12, 2001.
Application No. 10/278,582 is a continuation-in-part of application No. 10/020,270, filed on Dec. 12, 2001.
(60) Provisional application No. 60/486,304, filed on Jul. 11, 2003.

(51) Int. Cl. A61B 18/20

(52) U.S. Cl. 606/9; 607/89

(57) ABSTRACT

A method and apparatus for providing fractional treatment of tissue (e.g., skin) using lasers is disclosed. The method involves creating one or more microscopic treatment zones of necrotic tissue and thermally-altered tissue and intentionally leaving viable tissue to surround the microscopic treatment zones. The dermatological apparatus includes one or more light sources and a delivery system to generate the microscopic treatment zones in a predetermined pattern. The microscopic treatment zones may be confined to the epidermis, dermis or span the epidermal-dermal junction, and further the stratum corneum above the microscopic treatment zones may be spared.

This application (a) claims priority from U.S. Provisional Patent Application Ser. No. 60/486,304, filed on Jul. 11, 2003; and (b) further claims priority from and is a continuation-in-part of U.S. patent application Ser. No. 10/367,582, filed on Feb. 14, 2003, which claims priority from and is a continuation-in-part of both (i) U.S. patent application Ser. No. 10/279,093, filed on Oct. 22, 2002, and (ii) U.S. patent application Ser. No. 10/278,582, filed on Oct. 23, 2002, which claims priority from and is a continuation-in-part of both U.S. patent application Ser. No. 10/017,287, filed on Dec. 12, 2001, and U.S. patent application Ser. No. 10/020,270, filed on Dec. 12, 2001, all of which disclosures are incorporated herein by reference in their entireties.

### FIELD OF THE INVENTION

The present invention relates generally to methods and apparatus for providing medical or surgical treatment using optical energy, and in particular to a method and apparatus for providing fractional treatment of tissue (e.g., skin) using optical radiation.

### BACKGROUND OF THE INVENTION

Optical energy, particularly laser energy, is commonly used as a versatile tool in medicine to achieve desired outcomes in the tissue that is treated. For example, lasers have been used to treat common dermatological problems such as hypervascular lesions, pigmented lesions, acne scars, rosacea, hair removal, etc. Additionally, lasers are also used in aesthetic surgery for achieving better cosmetic appearance by resurfacing the skin and remodeling the different layers of skin to improve the appearance of wrinkled or aged skin. Generally, skin resurfacing is understood to be the process by which the top layers of the skin are completely removed by using chemicals, mechanical abrasion or lasers to promote the development of new, more youthful looking skin and stimulate the generation and growth of new skin. In laser skin remodeling, laser energy penetrates into the deeper layers of the skin and is aimed at stimulating the generation of and/or altering the structure of extra-cellular matrix materials, such as collagen, that contribute to the youthful appearance to skin. In traditional pulsed CO₂ laser resurfacing, the upper layers of skin may be completely ablated to a layer below the papillary dermis and there may be heat-diffusion-induced coagulation to several hundred micrometers below the original skin surface.

Generally, the desired effects on the skin are accomplished by laser-induced heating of the tissue. The induced heat results in thermal coagulation, cell necrosis, hemostasis, melting, welding, ablation and/or gross alteration of the extra-cellular matrix for specific temperature and heating time combinations. While using lasers for either skin resurfacing or remodeling, one of the important objectives has been to accomplish uniform treatment across the desired treatment area of the chosen skin site. Generally, particular care is exercised, either by the physician alone or by combining the physician's judgment with intelligence that is built into the dermatological system, to leave no tissue untreated in the targeted region of the skin. Whether one uses a broadly radiating pulsed beam of light or a focused laser that produces a relatively smaller spot size, the goal has been to expose the entire treatment area to the laser energy, heat the entire volume of tissue in the treatment area and bring about the desired change. It has been widely reported that such broad area treatment results in undesirable side effects such as intolerable pain, prolonged erythema, swelling, occasional scarring, extended healing times and infection.

Erbium lasers and CO₂ lasers usually cause a thermal treatment to a well-controlled depth. In contrast, yellow pulsed dye lasers designed for selective photothermolysis of microvascular lesions cause selective thermal treatment of microvessels of varying depths (See generally, Cutaneous Laser Surgery, edited by M P Goldman and R E Fitzpatrick and published by Mosby, 1999). Depending on the kind of laser used (CO₂, Erbium, etc.), the mode of usage (continuous wave or pulsed), the pulse width, energy density and power, different effects can be accomplished. FIG. 1 illustrates the prior art treatment of ablative laser skin resurfacing, where the target tissue 10 is primarily the epidermis 11. Typical laser skin resurfacing using prior art systems completely ablates the targeted epidermis 11.

An approach used in treating microscopic pigmented tissue targets is to take advantage of the selectively absorbed pulse of radiation. Selective photothermolysis is accomplished by site-specific, thermally mediated injury of microscopic, pigmented tissue or a particular chromophore, where the selective absorption is due to the laser absorption characteristics of the pigmented tissue and/or the particular chromophore. For example, the laser wavelength is typically chosen to target hemoglobin or a pigmented chromophore, such as melanin.

Typically in these cases, whether it is skin resurfacing or selective photothermolysis of anatomical structures or defects that are located deeper in the skin, a burn or an acute wound is created by the laser. For acute wounds, the skin heals by three distinct ‘response to injury’ waves, as illustrated in FIG. 2. The initial inflammatory phase 202 has a duration lasting minutes to days and seamlessly transitions into the cell proliferative phase 204, lasting 1 to 14 days. This cell proliferative phase is slowly replaced by the dermal maturation phase 206 that lasts from weeks to months (See, e.g., Clark, R. Mechanisms of cutaneous wound repair. In: Fitzpatrick T B, ed. Dermatology in General Medicine, 5^{th} Ed., New York, N.Y. McGraw-Hill. 1999. pp. 327-41, which is incorporated herein by reference).

In general, a direct correlation exists between the size of the injury and the time required for complete repair. However, the inflammatory phase 202 is a function of cellular necrosis, particularly epidermal (i.e., keratinocyte) necrosis, and a direct correlation exists between cellular necrosis and the inflammatory phase. Increased cellular necrosis, particularly epidermal necrosis, prolongs the inflammatory phase. Prolonging and/or accentuating the inflammatory phase may be undesirable from a clinical perspective due to increased pain and extended wound repair, and may retard subsequent phases of wound repair. The cause(s) of this prolonged inflammatory phase are not well understood. However, laser injuries are associated with early and high levels of dermal wound repair (e.g., angiogenesis, fibroblast proliferation and matrix metalloproteinase (MMP) expression) but delayed epidermal resurfacing (See, e.g., Schaffer et al., Comparisons of Wound Healing Among Excisional, Laser Created and Standard Thermal Burn in Porcine Wounds of Equal Depth, Wound Rep. Reg. v5 (1) pp. 51-61 1997, incorporated herein by reference). Unfortunately, most of the skin resurfacing efforts and selective photothermolysis treatments that affect large contiguous areas of chromophores result in a prolonged, exaggerated inflammatory phase 202 leading to undesirable consequences such as delayed wound repair. The prolonged inflammatory phase also leads to the pain experienced by most patients undergoing skin resurfacing procedures. Undesirable extended inflammatory response phase can be attributed to the bulk heating of the skin with little or no healthy tissue, particularly keratinocytes, left behind in the area where the skin was exposed to the laser energy. Particularly when uniform treatment is desired and the entire target tissue volume is exposed to laser energy without sparing any tissue within the target volume, pain, swelling, fluid loss, prolonged reepitheliazation and other side effects of dermatological laser treatments are commonly experienced by patients.

Many systems have been devised to minimize epidermal necrosis. One such approach includes cooling the epidermal surface using plastic bags filled with ice placed on the skin surface for a short while (about five minutes), compressed freon gas used during irradiation, or chilled water spread directly on the area being irradiated. Some of these methods are described in, for example, A. J. Welch et al., “Evaluation of Cooling Techniques for the Protection of the Epidermis During ND-YAG Laser Irradiation of the Skin,” Neodymium-YAG Laser in Medicine, (Stephen N. Joffe ed. 1983). Various devices and approaches have been proposed to treat dermal tissue regions without damaging the epidermal regions. One approach to minimize bulk heating of the skin is described in U.S. Pat. No. 6,120,497. In this approach for treating skin wrinkles, the dermal region is targeted in order to elicit a healing response to produce unwrinkled skin, and the epidermal region above the targeted dermal region is simultaneously cooled. In another example, U.S. Pat. No. 5,814,040 describes cooling an epidermal tissue region while performing selective photothermolysis of selected buried chromophores in biological tissues using a laser. This cooling procedure is known as dynamic cooling. As illustrated in FIG. 3, an epidermal tissue region is cooled by spraying a cryogen 302 on the surface of the epidermis 11 to establish a predetermined dynamic temperature profile. The epidermal 11 and underlying dermal 12 tissue regions are subsequently irradiated (not shown) to thermally treat the dermal tissue region (i.e. the altered tissue region 304 ) while leaving the epidermal tissue region substantially undamaged.

Another approach to sparing the epithelium during laser procedures includes a laser system that delivers laser energy over a relatively large tissue surface area with the laser light focused in the dermis (See, e.g., Muccini et al., “Laser Treatment of Solar Elastosis with Epithelial Preservation,” Lasers Surg. Med. 23:121-127, 1998). In this system, air is used as the coolant to maintain reduced temperature at the skin surface. Additionally, the optical device focusing the laser light also acts as a thermal conductor on the surface to help minimize surface temperature as air is flowed over the optical device to keep it cool.

All of these systems pose practical limitations because of the complexity added by the cooling system. Hence, there is a need for an improvement in the art for a system and method to treat the dermal region and avoid the complexities associated with cooling. In addition, all of these systems are macroscopic in nature, i.e., they expose the entire skin surface within the treatment region to laser irradiation (bulk heating) and cooling. These global treatments lead to an increase in clinical side effects and to an increase in healing time as described above. Hence, there is a need for an improvement in the art for a system and method to treat the dermal and epidermal regions that reduce the side effects associated with global non-ablative as well as ablative treatments. This reduction in side effects will allow physicians to increase the treatment intensity so that skin treatments can be provided more effectively.

When lasers act on the skin to cut, vaporize or coagulate tissue there are several ‘zones’ of tissue damage that surround the spot where the impact of the laser energy is the highest, i.e., the treatment zone where the tissue volume is necrosed either completely or to a level above a threshold, such as about 90% or more of the cells being necrosed. These zones are illustrated in FIG. 4. Usually, the temperature in the necrotic zone 402 has reached a value greater than about 70° C., and the tissue, whether it is made up primarily of cells, keratinocytes and their derivatives or collagen, is necrosed or denatured, respectively. The center of the necrotic zone is typically close to the center of the treatment beam. For heating times on the order of about 1-10 milliseconds, cell necrosis, coagulation and protein denaturization will occur in a range of or above about 65-75° C. Immediately adjacent to the area of necrosis is a thin thermal coagulation zone of tissue clumping (not shown), where denatured proteins have formed an area that contains necrotic cells, matrix, and cellular debris. Surrounding this zone is a larger zone of thermally-altered but viable tissue or a Heat-Shock Zone (HSZ) 404 in which proteins and cells have been heated to supra-physiologic temperatures over a short time, but a significant percentage still remain viable. In portions of this HSZ, the volume of the tissue is exposed to temperature typically in the 37° C. to 45° C. range—a range in which approximately 100% of the cells survive the treatment. The dimensions of these zones depend on various laser parameters (such as, wavelength, pulse duration, energy density, etc.), thermal and optical properties of the tissue components, and ambient temperature. Recent data indicate that the HSZ has special significance for subsequent biologic effects (See, e.g., Capon A. and Mordon S. Can thermal lasers promote wound healing? Am. J. Clin. Dermatol. 4(1):1-12. 2003, which is incorporated herein by reference). For illustrative purposes, the demarcation between the different zones is shown as an abrupt change. However, one skilled in the art would understand that the change from one zone to another is not abrupt, but gradual. Outside of the thermally-altered/HSZ 404, essentially unaltered healthy tissue 406 exists. Necrotic zone 402 and surrounding HSZ 404 together form a volume of thermally-altered tissue 408. Temperatures in the tissue above about 100° C. may cause steam to form in the tissue, which may cause disruptive effects.

Heat shock in the thermally-altered zone 404 triggers multiple signaling pathways that induce both cell survival and programmed cell death. The final outcome as to whether a cell lives or dies is believed to depend on the ‘acquired stress tolerance’ of the surrounding tissue. Mild heat shock followed by a period of recovery makes cells more resistant to subsequent severe heat shock and multiple other stresses. This is achieved via the activation of cell survival pathways (i.e., extracellular signal-regulated kinase, ERK, and akt kinase) and the inhibition of apoptotic pathways (i.e., Jun terminal kinase, Fas, caspase-8 and others) via heat shock protein (i.e., HSP72) mediated signaling events (See, e.g., Gagai V L and Sherman M Y, Interplay between molecular chaperones and signaling pathways in survival of heat shock. J. Appl. Physiol. 92:1743-48. 2002, which is incorporated herein by reference).

In conventional skin resurfacing and selective photothermolysis of contiguous chromophore regions, the laser exposed tissue is dominated by the necrotic treatment zone instead of the viable, heat shock zone. In fact, such conventional treatments are designed to cover the target tissue in the plane of the skin completely with overlapping necrotic zones so that no target tissue is left unexposed to laser energy. In contrast to conventional treatments, to promote the cell survival pathways and inhibit the apoptotic pathways, it is desirable to have the viable tissue be more prevalent in the laser exposed tissue compared to the necrotic zone. There is an unmet need for a laser treatment that enhances the proportion of a viable tissue portion in the target tissue volume.

### SUMMARY OF THE INVENTION

In general, the present invention features a method for treating either existing medical (e.g., dermatological) disease conditions or for improving the appearance of tissue (e.g., skin) by intentionally generating a pattern of thermally altered tissue surrounded by unaltered tissue. The thermally altered tissue may include a necrotic zone. This approach offers numerous advantages over existing approaches in terms of safety and efficacy. This invention minimizes the undesirable side effects of pain, erythema, swelling, fluid loss, prolonged reepithelialization, infection, and blistering generally associated with laser skin resurfacing. Another aspect of this invention is to stimulate the tissue's wound repair system, by sparing healthy tissue around the thermally altered tissue, whereby the repair process is more robust. Yet another distinguishing feature of this invention is to reduce or eliminate the side effects of repeated laser treatment to tissue by controlling the extent of tissue necrosis due to laser exposure.

One aspect of the present invention is a method for achieving beneficial effects in a target biological tissue comprising treating the target tissue using optical radiation to create one or more “microscopic” treatment zones such that the aspect ratio of the necrotic zone width to the necrotic zone depth is above about 1:2, preferably above about 1:4, and the treatment zones are created by a predetermined treatment pattern. Another aspect of this invention is a method for achieving beneficial effects in skin tissue comprising treating the skin by exposing a targeted part of the skin tissue to optical radiation to create one or more microscopic treatment zones such that the volume of the target tissue that remains unaffected by the optical radiation is controlled, and further that the ratio of the sum of the treatment zone volumes to the target tissue volume is less than one.

In one aspect of the invention, the microscopic treatment zones are created by using lasers with wavelengths in the range of 0.4 to 12.0 μm, directing the laser radiation to a targeted region in the skin, and creating microscopic treatment zones of necrotic tissue. These microscopic treatment zones could be in the epidermal or dermal regions or originate in the epidermal region and continue into the dermal region of the skin. In some embodiments, the upper layers of the epidermis, such as the stratum corneum, are spared and left substantially intact. The individual microscopic zones could have the shape of a cylinder, sphere, or any other shape that could be generated by an appropriate combination of wavelength, pulse duration, pulse width, beam profile, pulse intensity, contact tip temperature, contact tip thermal conductivity, contact lotion, numerical aperture of the focusing elements, optical source brightness, and power. Individual microscopic treatment zones are generally columnar in shape, which is beneficial for healing purposes. The microscopic treatment zones could be between 10 and 4,000 μm in the propagation direction of the beam (depth) and between 10 and 1,000 μm in the direction perpendicular to the beam (diameter).

Another specific aspect of this invention is a method of creating the microscopic treatment zones of necrosed tissue that allows viable tissue to be interspersed between the microscopic treatment zones thereby enabling the skin to mount a more robust repair response.

This invention also relates to an apparatus for treating common medical conditions by treating a target tissue volume in the skin with optical energy and creating one or more necrotic zones such that the aspect ratio of the necrotic zone diameter to the necrotic zone depth is at least about 1:2, and the necrotic zones are created by a predetermined treatment pattern. Another aspect of this invention relates to an apparatus that exposes a targeted part of the tissue to optical radiation to create one or more thermally altered treatment zones such that the volume of the target tissue that remains unaltered by the optical radiation is controlled. Further, the ratio of the sum of the thermally altered zone volumes to the target tissue volume is less than or equal to one.

Yet another aspect of this invention is an apparatus that provides the predetermined treatment pattern comprising at least one source of optical radiation and a delivery system operably coupled to the source and configured to direct the optical radiation to a volume of tissue in a predetermined pattern. The predetermined treatment pattern comprises a plurality of discrete microscopic treatment zones, wherein a subset of the plurality of microscopic treatment zones include individual discrete microscopic zones comprising necrotic tissue volumes having an aspect ratio of at least about 1:2. The source of radiation may include one or more lasers, flashlamps or LEDs. The delivery system may include various optical systems and/or scanner systems, such as lens arrays and galvanometer-based scanners, respectively.

### BRIEF DESCRIPTION OF THE DRAWINGS

These and other features, objects and advantages of the present invention are more readily understood from the following detailed description in conjunction with the accompanying drawings, where:

FIG. 1 is an illustration of skin exposed to laser radiation using a prior art system for skin resurfacing.

FIG. 2 is a schematic showing the inflammatory, cell proliferative, and dermal maturation phase of normal cutaneous wound healing.

FIG. 3 is an illustration of skin exposed to laser radiation using a prior art system for skin remodeling.

FIG. 4 is a schematic showing the different zones in a piece of skin exposed to laser radiation and consequent heat treatment.

FIG. 5 is an illustration of laser resurfacing using a prior art system.

FIG. 6 is an illustration of embodiments of the present invention.

FIGS. 7, 8 and 9 are schematics of the different thermally altered zones created by the incorporation of this invention.

FIGS. 10 and 11 illustrate different embodiments of this invention.

FIGS. 12a-12h illustrate various microscopic treatment zone shapes in accordance with various embodiments of the invention.

FIGS. 13a-13c and 14a-14g are graphical representations of different thermally altered zones created by various embodiments of the invention.

FIG. 15 is a schematic illustrating an embodiment of an apparatus for practicing the invention.

FIG. 16 shows an embodiment of the control system of the inventive apparatus.

FIG. 17 shows an embodiment of the optical system of the inventive apparatus.

FIG. 18 shows an embodiment of the delivery system of the inventive apparatus.

FIG. 19 is an illustration of a method of using of the inventive apparatus.

FIGS. 20, 21a, 21b and 22-24 are embodiments of systems for practicing the present invention.

FIGS. 25a and 25b show histological results from laser treatments applied utilizing embodiments of the present invention.

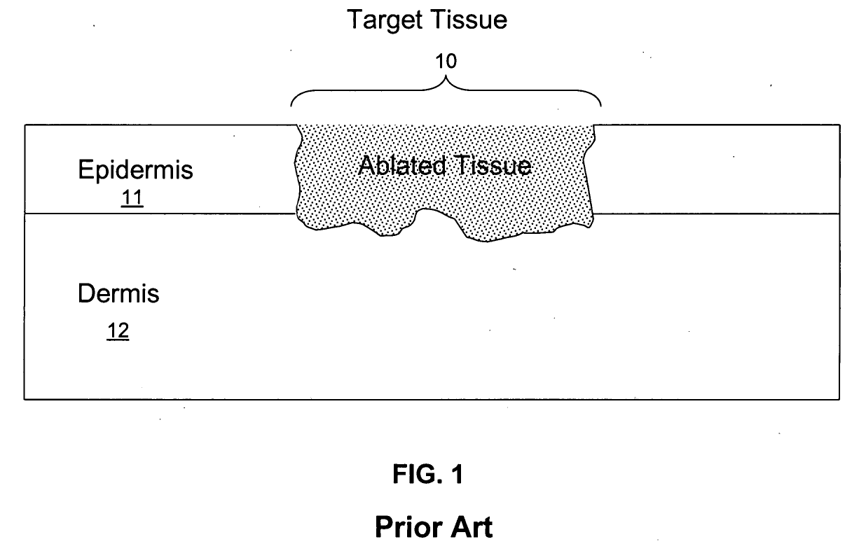

### DETAILED DESCRIPTION

Embodiments of the present invention provide a method and apparatus to increase the safety and efficacy of treating biological tissue with optical radiation, including dermatological treatments using lasers. Particularly, different embodiments of the present invention may be suitable to treat a variety of dermatological condition such as hypervascular lesions including port wine stains, capillary hemangiomas, cherry angiomas, venous lakes, poikiloderma of civate, angiokeratomas, spider angiomas, facial telangiectasias, telangiectatic leg veins; pigmented lesions including lentigines, ephelides, nevus of Ito, nevus of Ota, Hori's macules, keratoses pilaris; acne scars, epidermal nevus, Bowen's disease, actinic keratoses, actinic cheilitis, oral florid papillomatosis, seborrheic keratoses, syringomas, trichoepitheliomas, trichilemmomas, xanthelasma, apocrine hidrocystoma, verruca, adenoma sebacum, angiokeratomas, angiolymphoid hyperplasia, pearly penile papules, venous lakes, rosacea, wrinkles, etc. Embodiments of the present invention may be used to remodel tissue (for example, for collagen remodeling) and/or to resurface the tissue. While specific examples of dermatological conditions are mentioned above, it is contemplated that embodiments of the present invention can be used to treat virtually any type of dermatological condition. Additionally, embodiments of the present invention may be applied to other medical specialties besides dermatology. Other biological tissues may be treated with embodiments of the present invention, and in particular tissues with structures similar to human skin may be treated. For example, tissues that have an epithelium and underlying structural tissues, such the soft palate, may be treated using embodiments of the present invention. Skin is used in many places in this application as an example of one biological tissue that has been treated using embodiments of the present invention. However, it should be understood that the invention is not limited to skin or dermatology alone.

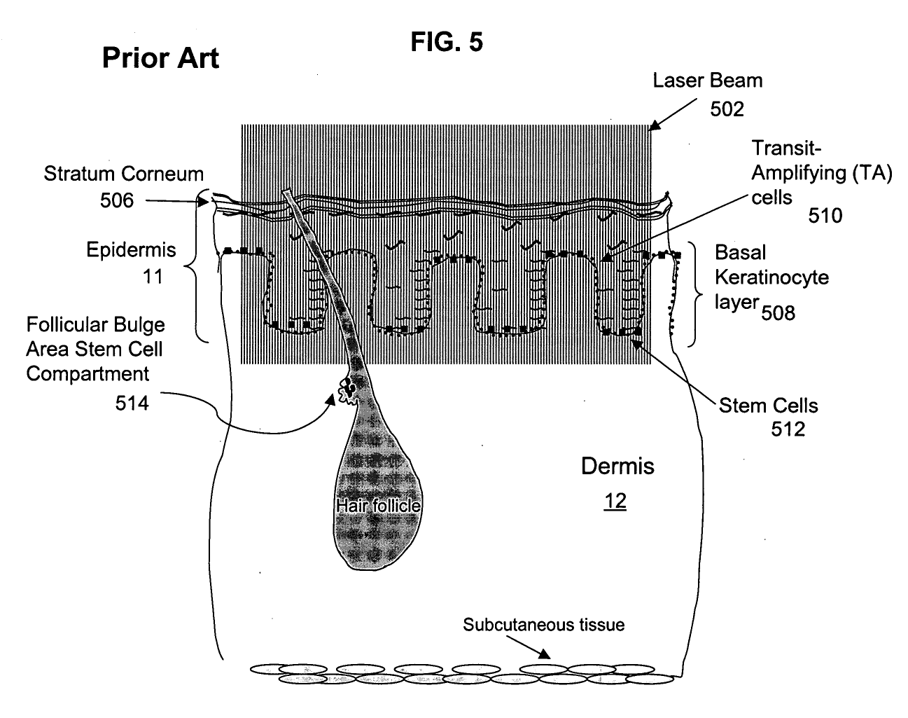

A primary mechanism of the present invention is the sparing of volumes of tissue within a larger tissue treatment area. In other words, leaving healthy tissue between and around necrotic treatment zones and HSZs has a number of beneficial effects that are exploited by various embodiments of the present invention. If the HSZs surrounding adjacent necrotic treatment zones are appropriately spaced and/or epidermal injury is limited, the viable tissue bordering thermal coagulation zones will be subjected to less inflammation from the products of cell death, thereby favoring cell survival over apoptosis. These areas will be better able to mount reepithelialization and fibro-proliferative and subsequent remodeling phases of wound repair. One important reason for this effect is that HSZs and bordering spared tissue contain subpopulations of stem cells responsible for repopulating the epidermis (See, e.g., Watt F, “The Stem Cell Compartment in Human Interfollicular Epidermis”, J Derm. Sci., 28, 173-180, 2002, which is incorporated herein by reference). In humans, stem cells reside in two locations in the skin: 1) in focal clusters of the basal keratinocyte layer, in contact with basement membrane components and, 2) in the follicular bulge area of the pilosebaceous unit. The basal keratinocyte layer of the epidermis typically contains a low population of these stem cells 512 interspersed with large numbers of transit-amplifying (TA) cells 510 that are directly derived from stem cells. Interfollicular epidermal stem cells tend to cluster at the bases of rete ridges in acral areas and at the tips of dermal papillae in non-acral skin. The follicular stem cell compartment 514 has been shown to possess the ability to repopulate the interfollicular epidermal surfaces when required under certain conditions. Such conditions include severe burns, large split-thickness epidermal injuries and cosmetic surgical procedures (e.g., ablative laser resurfacing, chemical peel, dermabrasion, keratotomy, etc.) that denude the epidermal layer, leaving no epidermal stem cell populations. Such denuding of the epidermal layer is illustrated in FIG. 5 by the large size of the laser beam 502 treating a large area of the epidermis 11. In fact, it is well known that CO₂ resurfacing results in prolonged reepithelialization when compared to steel scalpel or electrosurgical scalpel incisions even though laser wounds exhibit accelerated dermal healing (See, e.g., Schaffer et al., Comparisons of Wound Healing Among Excisional, Laser Created and Standard Thermal Burn in Porcine Wounds of Equal Depth, Wound Rep. Reg. v5 (1) pp. 51-61 1997, which is incorporated herein by reference). Reepithelization to repair such defects is delayed under these circumstances, because healing must occur from remaining follicular stem cell populations within the de-epidermized wound and from epithelial stem cells at the margins of the defect. If the wound is full thickness, extending down to the level of the pilosebaceous unit, then healing is delayed even further because epidermal healing occurs only from the margins.

The speed of epidermal reepitheliazation is directly proportional to the number and density of TA and stem cells. In the case of the follicular stem cell population, the average density of the bulge area compartment is dependent on the number of pilosebaceous units per unit of skin surface area. For the densest hair bearing skin (scalp) the number of adult human hair ranges between 100 and 500 per cm²; whereas surfaces such as the face have less than half that density. On the face, at least a two or three orders of magnitude greater density of epidermal stem cells exists versus follicular bulge stem cells based on the density of epidermal stem cell clusters that reside in the basal cell layer immediately above each dermal papilla in non-acral skin, where they are spaced every 10-100 μm.

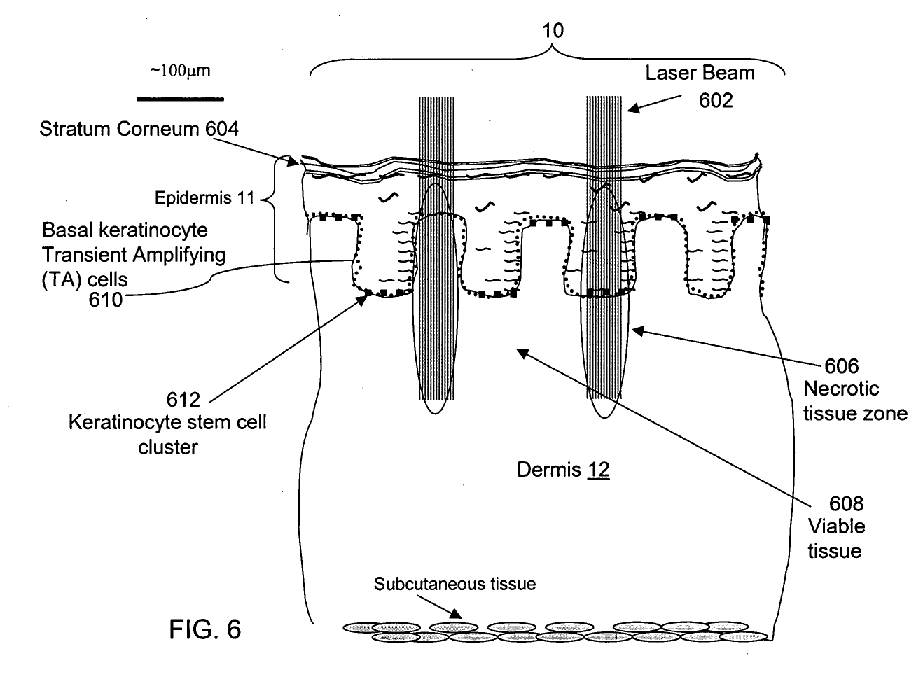

Fractional laser treatments according to embodiments of the present invention are illustrated in FIG. 6. If the entire volume of the target tissue is not treated but only a fraction of the tissue is treated by laser beams 602 thereby permitting the existence of viable tissue 608 (which typically includes HSZs and untreated, healthy tissue) between necrotic tissue zones 606, with multiple treatments, macroscopic areas of tissue regeneration will occur at the maximum rate within the surrounding micro-HSZs and spared epidermal surfaces, creating a ‘fractional wound repair field’ within the target treatment area 10. Such treatment may further include, but is not required to include, sparing the outermost layers of the epidermis, for example the stratum corneum, from significant damage. Such sparing of the stratum corneum promotes healing by maintaining the structural integrity and protective character of the stratum corneum. Fractional wound repair fields are fundamentally different from previous techniques because the areas of epidermal tissue that are spared between necrotic zones contain both epidermal stem cells 612 and TA cell populations 610. Thus, re-epithelization of necrotic zones proceeds rapidly with few or none of the side effects (i.e., pain, persistent erythema, edema, fluid drainage, etc.) observed after traditional resurfacing procedures. A small necrotic zone cross-section (e.g., less than about 250 microns in diameter for a circular cross-section) means that a significant number of stem cells and TA cells are relatively close to the center of the treatment zone throughout the depth of the treatment zone. This further speeds the healing response, such that substantially complete (e.g., greater than about 75% complete) re-epetheliazation typically occurs in less than about 36 hours post-treatment for necrotic zone cross-section widths in a range less than about 250 microns, and preferably for cross-sectional widths less than about 100 microns substantially complete re-epetheliazation occurs less than about 24 hours post-treatment. Re-epetheliazation typically occurs at a rate directly proportional to the cross-sectional width of the necrotic zone. As a further example, if the spacing between fractional beam treatment zones creates an average density (i.e. number of necrotic zones per unit surface area of the target treatment area 10) of 500 necrotic zones/cm² there are ample epidermal stem cells that remain for interfollicular resurfacing of both the necrotic zone itself and of the surrounding HSZs, if necessary. In addition, after fractional laser treatment, the follicular bulge stem cell population remains intact, so they may participate in wound healing and resurfacing, as needed. The density of treatment may alternately be described with a fill factor (i.e. surface area receiving radiation or necrosed divided by total surface area of the target treatment area 10), wherein a typical fill factor for embodiments herein may be between about 0.05 and about 0.95, and preferably between about 0.1 and about 0.5.

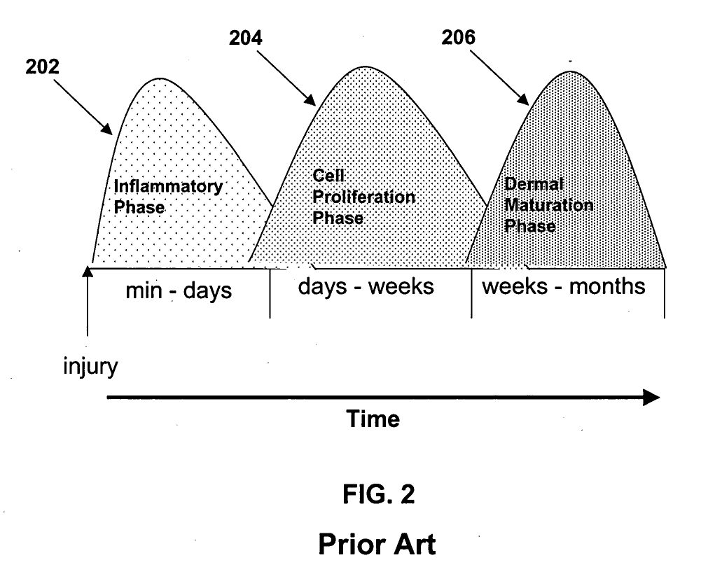

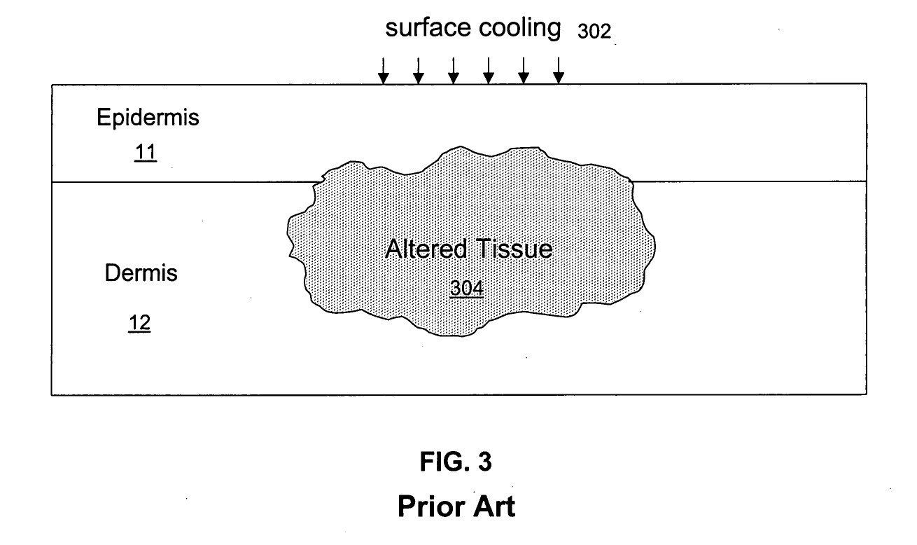

Chronic UV irradiation appears to trigger dysfunctional wound repair pathways in the skin that involve gradual replacement of normal epidermal and dermal structures with characteristic atrophy and accumulation of elastotic dermal matrix components (See, e.g., Kligman, “Prevention and Repair of Photoaging: Sunscreens and Retinoids”, Cutis. May 1989:43(5):458-65). Currently, reversal of photo-aging is attempted by imparting cutaneous injury that induces new dermal collagen formation. Such cutaneous injury could be accomplished using mechanical (e.g., dermabrasion), chemical (e.g., retinoids and acid peels), or laser surgical procedures. The expectation is that these cutaneous injuries will promote the normal fibro-proliferative responses of the upper reticular and papillary dermal compartments, and therefore yield rejuvenated skin. For example, U.S. Pat. No. 6,120,497 describes thermally injuring collagen in the targeted dermal region to activate fibroblasts. The fibroblasts in turn deposit increased amounts of extracellular matrix constituents. However, as discussed above with reference to FIG. 2, epidermal injury promotes the inflammatory phase, which inhibits the rejuvenative process. As can be easily imagined, dermabrasion, which is a mechanical surface ablation process, results in epidermal injury. Hence, while the currently used methods, which are mentioned above, for promoting normal fibro-proliferative response of the dermal compartments can yield rejuvenated skin, due to the epidermal injury that occurs with these processes, the rejuvenative process is compromised.

An objective of nonablative photorejuvination is to induce a thermal wound repair response in the papillary and upper reticular dermal compartments (approximately 100-400 μm below the surface of the skin) while sparing the epidermal compartment. To spare the epidermis, one typically uses low fluences (laser energy densities). Unfortunately, such low levels are generally inadequate to promote the kinds of stimulation that are required to cause the desired dermal effect. Thus, prior art approaches result in minimal efficacy. In most cases, minimal dermal matrix remodeling and minimal clinical responses (e.g., wrinkle reduction, retexturing, dyschromia reduction, and telangiectasia removal) are achieved by these procedures (See, e.g., Nelson et al., “What is Nonablative Photorejuvenation of Human Skin”, Seminars in Cutaneous Medicine and Surgery, Vol. 21, No. 4 (December), 238-250, 2002; Leffell D, “Clinical Efficacy of Devices of Nonablative Photorejuvenation”, Arch. Dermatol. 138: 1503-1508, 2002). Therefore, there is an unmet need for sparing the epidermal compartment, but achieving enough stimulation of dermal matrix remodeling to be clinically effective.

By creating isolated, non-contiguous (i.e. discrete) treatment zones having necrotic tissue surrounded by zones of viable (i.e. heat altered viable tissue and often untreated, un-altered healthy tissue) tissue that are capable of promoting healing, the present invention induces multiple sites of tissue regeneration to produce ‘micro-thermal wound repair fields’. We call this process fractional photo therapy, as fractional volumes of the target tissue volume are thermally altered, as opposed to the conventional treatments where the entire target volume is thermally altered or damaged. Each field is typically composed of thousands of individual thermally altered zones (i.e. HSZs and surrounding spared tissue units) that comprise “nodes” of wound repair. The healing mechanisms (e.g., stem cells and TA cells) of each node can be expected to expand beyond the volume of the node to merge with neighboring nodes, replace photo-aged tissue components (e.g., solar elastosis, microvascular ectasia, pigment incontinence, epidermal atrophy, and atypia), and produce complete coverage. Hence, there is a need for generating isolated, non-contiguous tissue volumes having treatment zones composed of necrotic tissue, surrounded by zones of viable tissue that are capable of promoting healing of the target tissue. The present invention meets this need.

Furthermore, some embodiments of the present invention protect the stratum corneum and uppermost layers of the epidermis from ablation, puncture or other significant damage. This is typically achieved by such means as choosing appropriate pulse energies and durations, and using a contact window placed against the tissue during treatment. For example, sapphire or diamond windows may be used for their high thermal conductivity and transparency to pertinent wavelengths. Additionally, choosing wavelengths that act on water as the primary or substantially only chromophore assists in limiting damage to the stratum corneum, as the stratum corneum typically includes relatively small amounts of water. The result of these embodiments is to maintain the integrity of the stratum corneum such that its physical structure is intact. This allows the stratum corneum to continue its normal function of protecting tissue underneath it from infection, dehydration, etc. For most tissue, water makes up a large part of the tissue such that water as a chromophore is typically contiguous throughout the treatment volume. In such tissue for embodiments using water as the primary chromophore, selective photothermolysis typically has little application, and it is the beam shape and parameters that define necrotic zones and that allow viable tissue to remain between necrotic zones. Contact windows are not required for all embodiments of the present invention. Non-contact windows may be used, such as, for example, windows set at a constant height above the tissue surface. Further, contact windows may be less than 100% transparent to the treatment beam wavelength, such as, for example, less than about 75% transparent. Additionally, contact windows may have low thermal conductivity. Such partially transparent and/or low thermal conducting contact windows may beneficially generate heat for use as part of a treatment.

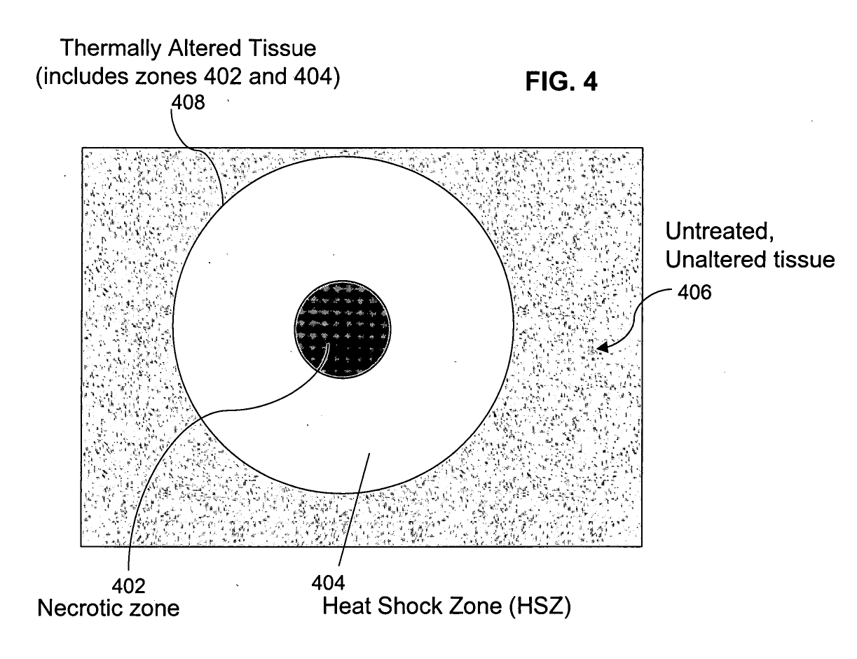

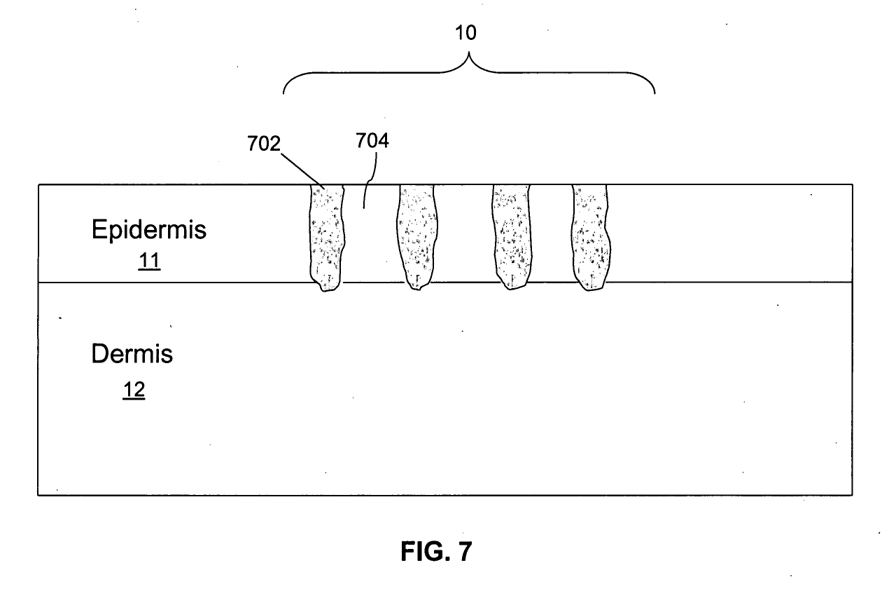

FIGS. 6 through 9 illustrate some embodiments of this invention. In FIGS. 6 through 9, target tissue 10 is the volume of tissue comprising thermally altered and unaltered tissue that is being addressed by the therapy. In FIGS. 6 and 7 the intended treatment is resurfacing of the skin so that the patient's skin looks younger and healthier. The objective is to remove a portion of the epidermis 11 and stimulate the rejuvenation process in the dermal region 12. As shown in FIG. 4, the thermally altered volume of tissue 408, comprises the treatment zone 402 and the HSZ 404. The thermally unaltered tissue 406 surrounds the thermally altered volume of tissue 408. The thermally altered volume of tissue 408 comprising the treatment zone 402 and the heat shock zone 404 (HSZ) is further illustrated in FIGS. 7 through 9. For illustrative purposes the boundaries between the treatment zone 402 and the HSZ 404 are clearly marked. One skilled in the art would understand that the treatment zone 402 is made up of tissue that has been almost completely necrosed (e.g., such that greater than about 75%, and preferably greater than about 90%, of the originally viable cells in the zone are necrosed post-treatment) and the HSZ 404 is made up of substantially viable tissue that has been thermally altered (e.g., such that greater than about 50% of the cells in the zone that were viable before treatment are still viable). Treatment zone 402 is made up of tissue that has lost its inherent biological activity and has typically experienced temperatures higher than about 70° C. for a significant length of time (i.e. greater than about 1 millisecond). HSZ 404 is the tissue volume surrounding necrotic zone 402, and HSZ 404 has typically been exposed to temperatures above 37° C. and up to as much as 55° C.-65° C., for typical heat exposure times of about 1 msec or less. This thermally altered tissue is viable and capable of mounting and assisting a robust tissue repair response. One skilled in the art understands that the boundary regions are not clearly defined in that there is typically a temperature gradient from the center of the necrotic zone outward, such that heating and the percentage of cell necrosis decreases from the necrotic zone 402 through the HSZ 404. The necrosis process is typically described by an Arrhenius-type model where thermal damage is cumulative, irreversible and linked to the time of exposure and heating rate.

FIG. 7 illustrates the situation where the necrotic zones 402 are predominantly in the epidermis 11, with viable tissue 704 between necrotic zones. FIG. 6 illustrates the effect of the inventive treatment where a significant portion of the keratinocyte stem cell cluster 612 and the basal keratinocyte transient amplifying cells 610 are spared. Again, one skilled in the art would understand that the treatment zones 402 and the HSZs 404 do not abruptly end at the epidermal-dermal junction, but are substantially in the dermis as well. It is likely that there will be a thermal spread into the dermis 12. The extent of the thermal spread is generally a function of the power, pulse width, repetition rate for multiple laser firings, and wavelength of the laser beam, the numerical aperture and focus depth of the optical system, and the thermal conductivity and temperature of the tip that could be placed in contact with the surface of the skin, all within the context of the scattering, absorption and thermal conductivity characteristics of the tissue.

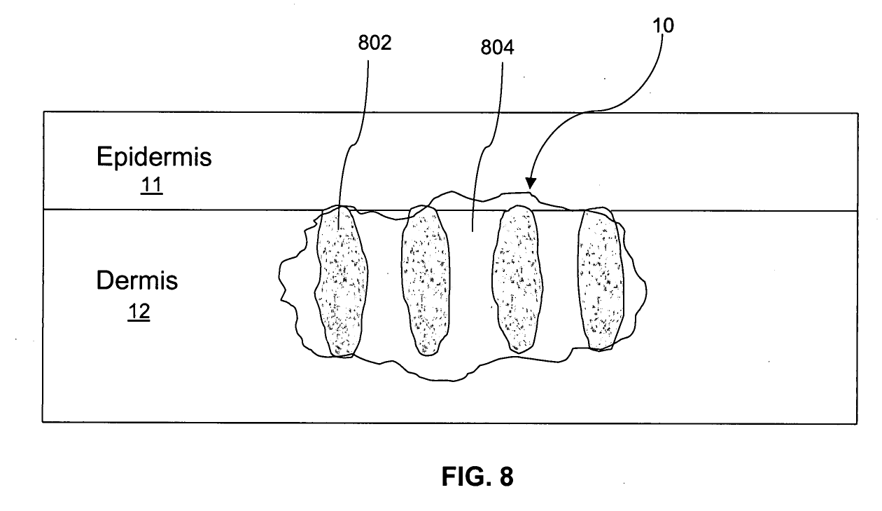

FIG. 8 illustrates a skin remodeling treatment where the target tissue 10 is the primarily in the dermis 12. Thermally altered tissue 802 is primarily confined to the dermis 12. Again, it is to be understood that it is likely that a thermal spread could occur in the epidermis 11.

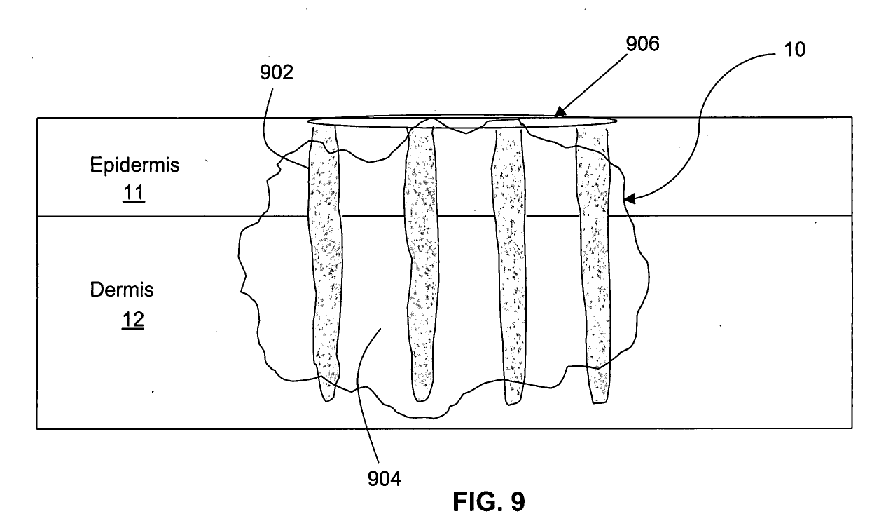

FIG. 9 shows where the thermally altered tissue 902 spans the epidermis 11 and the dermis 12. This illustrates the situation where one desires to have skin resurfacing, partial removal of the epidermis 11, and collagen shrinkage in the dermis 12. Additionally, FIG. 9 illustrates sparing the stratum corneum at the tissue surface in area 906.

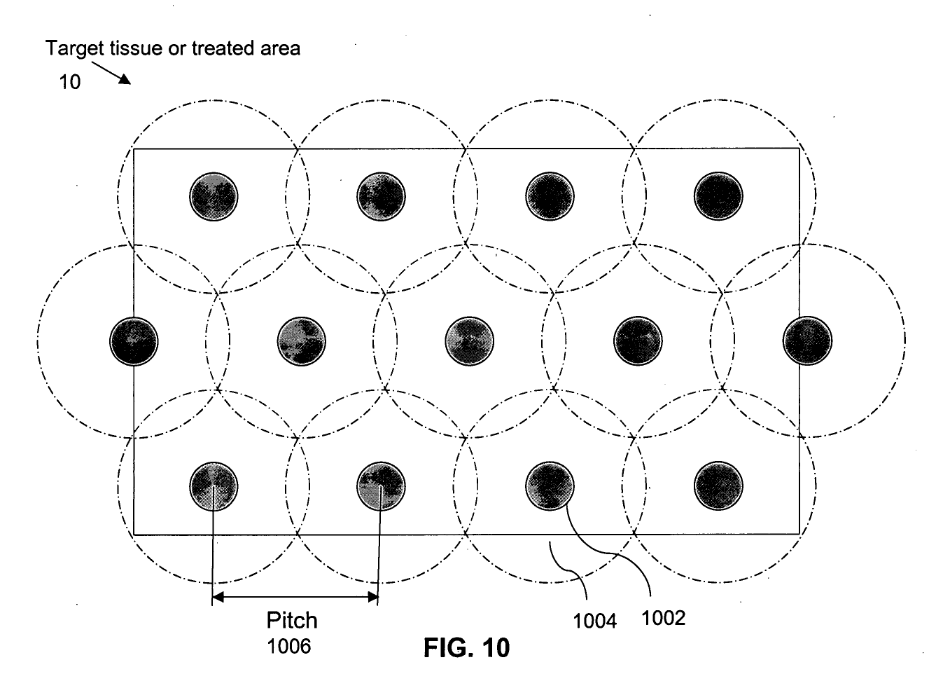

FIG. 10 shows an alternate embodiment of the present invention, where the heat shock zones 1004 overlap. The center of the target zones 1002 are separated by pitch 1006. If the pitch is less than the diameter of the HSZs 1004 then the HSZs overlap. These overlapping HSZs 1004 can be positioned such that, overall, the target tissue 10 is left with no thermally unaltered tissue. One way the HSZs 1004 can be made to overlap with each other is by adjusting where the laser beam lays down the spots (i.e. where the center of the necrotic zones 1002 are placed). For example, if two spots are within less than about 100 microns of each other, there will typically be such overlap. If two or more treatment zones 1002 are designed to lie in close proximity to each other and if the spots are laid down in quick succession, then the net increase of temperature due to closely spaced treatment zones may be sufficient to increase the size of individual HSZs 1004. In this type of treatment, it is important for the treatment zones that are contributing to the creation of the HSZs to be created in a time short enough to prevent thermal diffusion from removing thermal energy from the adjacent treatment regions that are contributing thermal energy to the creation of the spatially enhanced HSZ. Another method uses a combination of thermal diffusion and overlap of thermal energy to create spatially enhanced HSZs. It should be noted that the thermal diffusion constant depends on the chemical constituents of the tissue (i.e. bone, fat, tendon, etc.), dimensions of the cell structures, water content and heat dissipating blood flow. Consequently, the thermal diffusion constants are different in the avascular epidermis and highly vascularized dermis. An alternative way to overlap the HSZs 1004 will be to make the HSZ 1004 significantly larger than the treatment zone 1002. One approach to make the HSZ 1004 larger than the treatment zone 1002 is to generate the desired treatment zone 1002 using high energy densities, such that high temperature regions are created. These high temperature zones would then spread the thermal energy over a larger volume that would result in a larger HSZ 1004. It may be detrimental to various treatments to have the treatments zones so close that they overlap, as this may cause blistering and/or significant clefting or lift-off at the dermal-epidermal junction.

As illustrated in FIG. 11, one important aspect of this invention is skin laser treatment that intentionally leaves behind healthy, substantially unaltered tissue 1102, such that the substantially unaltered tissue 1102 helps in skin remodeling and wound repair of the treatment zones 1104. FIG. 11 depicts target tissue 10 made up of necrotic zone 1104, HSZs 1106, and thermally unaltered tissue 1102. Thermally unaltered tissue 1102 typically does not receive any laser light directly from the treatment system. Laser light from the treatment system typically radiates the tissue surface only within necrotic zone 1104. As described in further detail below, the shape and size of the treatment zone 1104 and the consequent HSZ 1106 can be controlled by choosing the appropriate laser parameters. The volume of the unaltered tissue 1102 and the spacing between zones of thermally affected tissue 1104 and 1106, and thermally unaltered tissue 1102 can also be controlled by choosing the appropriate treatment parameters and treatment beam spacing. Additionally, the stratum corneum may be protected and maintained intact, or it may be ablated or damaged during treatment, depending on the desired effect. In various embodiments described below, necrotic zones and HSZs may be created in a predetermined pattern (e.g., a polygonal grid pattern, a circular pattern, a spiral pattern, a dot matrix, dashed lines, dashes, lines, etc.) or in a random pattern. If a predetermined pattern is used, the pattern may be uniform, non-uniform or partially uniform in shape and/or spacing, and the individual treatment volumes may be substantially uniform, substantially non-uniform or partially uniform in shape and size. Within a larger treatment area, subsets of necrotic zones and HSZs may be overlapping to create clusters or lines of necrotic zones, with areas of healthy tissue between clusters or lines (e.g., dashed lines less than about 1 centimeter). Additionally, different embodiments may include the use of treatment beams of optical radiation that are interleaved or sequentially, simultaneously or randomly generated to create the predetermined or random patterns.

### Controlling the Shape and Depth of the Treatment Zones

A wide variety of treatment zones of varying depths and shapes can be created using the optical systems described herein. The shape of the region of necrosis created in the tissue, and the shape of the HSZ surrounding it can be adjusted using appropriate combinations of the laser parameters.

The shape of the treatment zones is affected by a combination of the wavelength of the light, the size and shape of the optical beam, the optical focusing, the flatness of the skin surface and the laser pulse parameters (e.g., energy, duration, frequency). The wavelength of the light selects values for the optical absorption strength of various components within the tissue and the scattering strength of the tissue. These optical transport parameters determine where the light energy travels in the tissue, and serve to partially determine the spatial temperature profile in the tissue. The size and shape of the optical beam and the focusing or numerical aperture of the laser determines gross propagation properties of the beam inside the tissue. Size (e.g., diameter for a circular beam shape or cross-sectional width for a polygonal or irregularly shaped beam) and shape of the optical beam, particularly as the optical beam enters the tissue, typically affects the shape of the resulting necrotic zone. For example, a polygonal cross-section for the optical beam may produce a polygonal columnar necrotic zone, and a circular optical beam cross-section typically produces a circular or oval necrotic zone cross-section. Cross-sectional width for beam shape means the smallest distance across the cross-section in a line that includes the center of the cross-section. Cross-sectional width includes diameter, as diameter is simply a specific instance for a circular beam cross-section. Focusing, or numerical aperture (N.A.), is a significant factor for determining the ratio of the surface temperature of the tissue to the peak temperature reached in the most intensely affected zone. Embodiments of the present invention may include varying or alternating focal depths for one or more optical beams impacting a give treatment zone. For example, such embodiments may include multiple optical beams focused to different depths, or the may include a single beam that is focused to varying depths within a treatment zone. The magnitude of the temperature profile is determined in part by the laser pulse energy.

The shape and size of a treatment zone is roughly determined by the region of the tissue that reaches a temperature in the appropriate temperature range for that treatment. Thus, for example, a particular treatment may be divided up into zones A-D. For example, zone A might be the region where the peak temperature reaches 75° C. or higher, zone B might be the region where the peak temperature lies in the range 62-75° C., zone C might be the region where the peak temperature lies on the range 45-62° C., and zone D might be the region where the peak temperature lies below 45° C. These temperature ranges may be set by a practitioner of the present invention to define regions where particular desirable (or undesirable) effects are dominant in the tissue, according to the earlier description of the influence of heat on human tissue. Typically, for temperatures above about 70° C. for heating durations of greater than about 1-2 msec, tissue will coagulate and necrose and proteins will be denatured. Heat shock zones will typically be created for tissue temperatures less than about 45-50° C. One of ordinary skill will recognize (a) that more or fewer zones may be defined with different temperature ranges in characterizing the ‘fractional’ aspects of this invention, and (b) that the definition of a treatment zone may be based on tissue biochemistry rather than on the peak temperature. For example, an area having cell necrosis to a level of greater than about 75%, and preferably greater than about 90%,. of all cells being necrosed is considered herein as a necrotic zone. Necrosis may be determined by a variety of histological processes, including for example, hematoxylin and eosin (H & E) stains or nitro-blue tetrazolium chloride, a lactate hydrogenase (LDH) activity stain. Loss of birefringence due to thermal denaturation of collagen may be evaluated, for example, using cross-polarized light microscopy.

An example of the control of heat affected zones using the laser pulse energy is provided by the case of a collimated or weakly diverging incident laser beam. In this situation, the beam spreads out inside the tissue, and creates treatment zones that resemble concentric shells centered on the point of entry of the laser into the skin. The ‘treatment’ in each of these treatment zones is defined by the temperature range achieved in the specific zone. In the absence of skin surface cooling, the zones may well extend out to the skin surface and indeed in this case some part of the skin surface usually lies in the most intensely affected zone (i.e. the zone with the highest temperature rise). If the laser pulse energy is small, these zones do not penetrate deeply into the skin. For weak laser pulse energy, only the least intense treatment zones (e.g. zones C and D of the previous paragraph) will be created. The zones for the more intense treatments do not exist for weak laser power. For higher laser pulse energy the treatment zones penetrate more deeply into the skin, and zones of increasing treatment levels (e.g. zone B and then A of the previous paragraph) are created close to the surface. As the laser energy is increased further the smaller zones close to the surface expand to greater depths in the skin.

A further example of the control of thermally altered zones (and especially necrotic zones) using the laser power and wavelength and external focusing is provided by the case of a tightly focused incident laser beam. In this situation, the effective beam diameter tends to reduce inside the tissue, reaching its smallest diameter (effective “focus”) at a depth given by the balance between focusing and optical scattering. At levels deeper than the actual focus the beam spreads out rapidly. In the wavelength region around 1450 nm, the absorption depends strongly on the wavelength. For this example, we select the wavelength so that the absorption depth is equal to the depth of the actual focus. Further, the focal length of the incident laser beam is selected so that the on-axis intensity of the laser beam increases for increasing depth below the tissue surface, peaks at or near the actual focus, and then decreases.

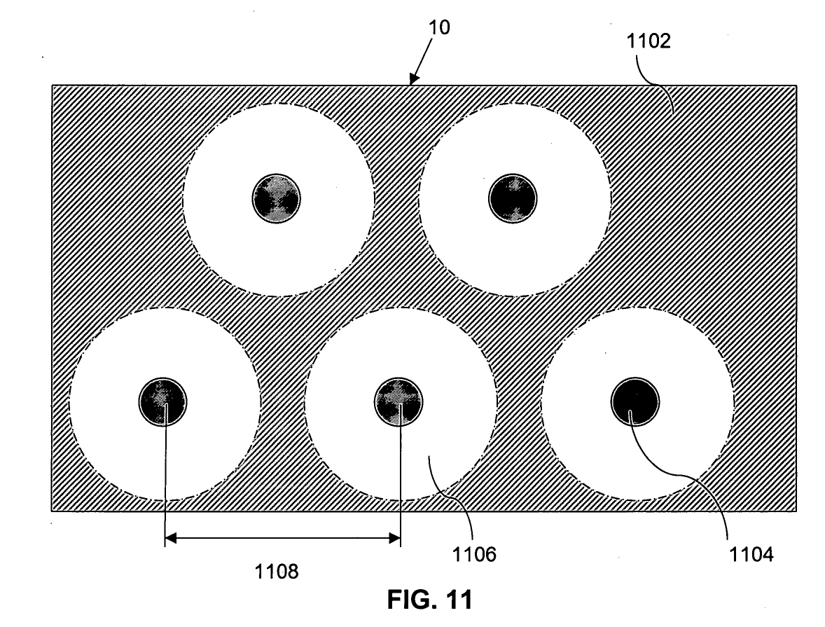

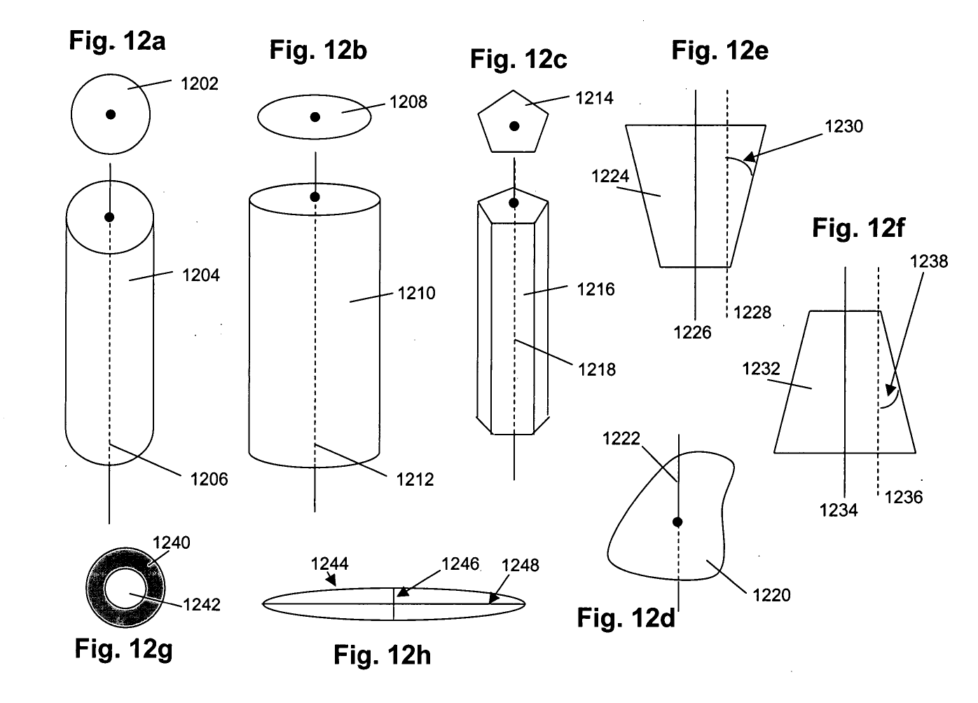

Under these circumstances in this example, the following beneficial result is obtained—the necrotic zones, as well as typically the surrounding HSZs, are substantially columnar regions or columnar shells centered about the actual focus. By substantially columnar we mean a shape that is approximately cylindrically symmetric along the optical axis of the treatment and deeper into the tissue than it is wide. It includes shapes such as spheroidal (round-ish), ellipsoidal (fat cylinder), cylindrical (right cylinder), bispherical (pinched cylinder), or conoid (tapered). Other words to describe the columnar shape might be cigar-like, prolate-, right-cylindrical, or conical. Substantially columnar as used herein includes circular (e.g., FIG. 12a (1202)), oval or elongated (e.g., FIG. 12b (1208)), irregular (e.g., FIG. 12d (1220)) or polygonal (e.g., FIG. 12c (1214)) shaped cross-sections (i.e., cross-sections perpendicular to the optical axis of the treatment beam). As illustrated in FIG. 12g, the cross-section may also be annular in shape, such that the necrotic zone 1240 surrounds a viable tissue portion 1242. Substantially columnar necrotic treatment zones are further described as elongated in the direction parallel to the optical axis of treatment. Substantially columnar further includes necrotic zones with sides or lateral aspects that are substantially parallel to the optical axis of treatment, although this includes sides that are up to about 40° tilted (e.g., angle 1230 in FIG. 12e or angle 1238 in FIG. 12f) in either direction with respect to the optical axis of treatment. The term substantially columnar does not necessarily imply symmetry below and above the actual focus, and further includes sides that are bulged or indented. For example it includes a shape which is a half-spheroid above the actual focus and a tapered conoid below the actual focus.

At low laser pulse energy, only one zone is created, that is, the zone corresponding to the weakest treatment (e.g. Zones D or C). For the parameters given in this example, this shape will be substantially columnar. For larger pulse energy, the zones are longer and a little wider. At still larger pulse energy, the new zones corresponding to more intense treatments appear as small regions centered on the actual focus. At still larger pulse energy, the zones all increase in size. And so on, until at the highest laser pulse energies, the most intensely affected zone created is a zone corresponding to over-treatment (e.g., charring and/or ablation) of the tissue.

In each of these examples the temperature history of the tissue is typically relevant. For short laser pulses, where heat transport is not strong during irradiation, the temperature at any location in the tissue rises to its peak value, (thus determining the zone type for that location), and then decays back to ambient temperature as a result of heat transport. The rate at which the temperature decays depends on several factors, including the water content of the tissue, the degree of vascularization of the tissue, the physical size and shape of the treatment zones and the actual temperature profile in the tissue. There is evidence that the rate of rise of the temperature can significantly affect the response of the tissue to the increased temperature. A rapid rise may cause a more intense reaction than a slow rise. Also a previously treated region may respond differently from a previously untreated region. To the extent that the actual temperature history is significant, the laser pulse length can be adjusted to control this parameter. For reproducible results, a preferred embodiment selects a pulse length for which the effects of a slow temperature rise or possible thermal pre-treatment are avoided. Separation between thermally-altered zones avoids adjacent treatment zone heating. This is generally achieved for shorter pulse lengths (i.e. less than about 25 msec) for necrotic zone cross-sectional widths less than about 150 microns. However, this recommendation for the pulse length should not be construed as a limitation on the invention.

The optical properties of the tissue may vary with temperature and biochemistry. For example it is well-known that optical absorption features in the skin are known to vary with temperature. Also, optical scattering in the dermis is believed to decrease and then increase with increasing thermal denaturation of collagen. The use of all these effects by adapting the laser parameters to account for them and take advantage of them is within the scope of the present invention.

This type of control has been verified using computer modeling and also by experiments on human tissue. Based on this modeling and experiments, it is possible to set the laser irradiation parameters to achieve either or both of epidermal treatment and deeper dermal treatment. For collimated or diverging incident beams the shell zones lie close to the skin surface and often touch it, and for tightly focused incident beams, columnar zones can be centered well below the skin surface. In particular, the shape of the treatment zones can be varied among all the shapes described above, by adjusting one or more various parameters such as the wavelength, external focus power (in diopters) or numerical aperture, external pressure on the skin, the presence or absence of a contact plate at the skin surface, the laser pulse energy and laser pulse duration, laser beam shape and size, and the repetition frequency of pulses. Some embodiments take advantage of the temperature-based shifts of the absorption features in skin to control precisely the shape and extent of the treatment zones.

### Modeling Guidance

We provide here a model for use in practicing this invention. The general shape of the heated region is approximated by a model in which the RMS radius of the heated region as a function of t and z is
ρ²=(R²−w²)(z/f−1)²+w²+b²z³+4Dt
where z is the depth below the surface, t is time since the optical pulse began, R is the radius of the beam at the skin surface, f and w are the location and size, respectively, of the beam waist in the absence of scattering. Scattering and thermal diffusion are represented by the last two terms, where b²=⅓μ(1−<cos θ>), and D is the thermal diffusivity within the tissue. μ is the scattering coefficient, θ is the scattering angle, and <cos θ> is the average value of the cosine of the scattering angle. Within this model the temperature rise in the tissue at the end of the laser pulse as a function of r is

$$T = \frac{α E {ⅇ}^{- α z}}{C π {ρ}^{2}} {ⅇ}^{- {( r / ρ )}^{2}}$$

where α is the optical absorption of the tissue, E is the laser pulse energy that enters the skin, C is the specific heat of the skin, and ρ is evaluated at the end of the optical pulse where t=τ. Within this model, the boundaries between treatment zones may be based on the magnitude of the temperature at the end of the pulse.

Along the optical axis of the beam (i.e., r=0), the temperature profile is determined by the competition between reduced beam diameter in tissue and optical absorption. The actual focus of the beam, where the beam waist p is smallest, typically occurs at a depth z₀ less than f, as a result of scattering. The actual beam waist is w₀=ρ(z₀) evaluated at the beginning of the optical pulse, where t=0. For weak absorption, the temperature is highest at depth z₀, whereas for strong absorption the heated region lies closer to the skin surface. There is therefore an absorption value for which the temperature rise below the skin surface is a maximum. It is given by
α_{peak-T}z₀=1

The ratio of the temperature rise at the surface to the temperature rise at depth z is

$$\frac{T ( 0 )}{T ( z )} = {{ⅇ}^{- α z} ( \frac{ρ ( z )}{R} )}^{2} \approx \frac{{w}^{2} + {b}^{2} {z}_{0}^{3}}{e {R}^{2}}$$

Given z₀, these equations indicate the optimal absorption and the beam parameters to use to select a suitable surface temperature rise. The wavelength is chosen to achieve a desired absorption based on target chromophore(s), whereas the relation between z₀ and the focal length f depends on the scattering, which the practitioner generally has minimal ability to control.

The shape of the treatment zones may be described by the location of the boundaries between treatment zones. Within this model, they are given by T(r,z)=constant.

$${( \frac{r}{{w}_{0}} )}^{2} = {( \frac{ρ}{{w}_{0}} )}^{2} [ {K}^{2} - α ( z - {z}_{0} ) - 2 ln ( \frac{ρ}{{w}_{0}} ) ]$$

where K is a constant such that the radius of the treatment zone boundary at z=z₀ is Kw₀. Note that whether the depth of interest z is deeper or shallower than the actual focus, the ratio ρ/w₀ is always greater than one. ρ/w₀ is therefore quadratic in z-z₀ near the actual focus, but may increase faster than this at greater distances from z₀. The boundaries predicted in this way are substantially columnar in the sense described above.

One of ordinary skill will recognize many of the assumptions that underlie the model. This model is informed by more detailed calculations involving optical refraction and diffraction, Monte Carlo light propagation and thermal diffusion in three spatial dimensions, and detailed reaction rates for biochemical processes in tissue. We therefore offer this model as a general guide to the practitioner of the invention in selecting appropriate parameters for the control of the various treatment zones.

After the optical pulse has terminated, the heat in the tissue continues to diffuse and raise the temperature of the surrounding tissue. There is usually more thermal energy in a treatment zone (e.g., necrotic zone or thermally altered zone) than the minimum required to raise the temperature of the tissue to the level to achieve the particular tissue condition for that zone. This extra energy is available to cause further tissue changes in the surrounding regions. Thermal diffusion and other known mechanisms cause this transport to occur. Thermal diffusion therefore has the effect of expanding the treatment zone by an amount that depends on its excess thermal energy and the radius of the lesion. The net effect of thermal diffusion is that it expands the treatment zone and tends to make the treatment zones more spherical. The effect is generally small unless very large amounts of excess energy are applied to the tissue or the lesion has a large diameter.

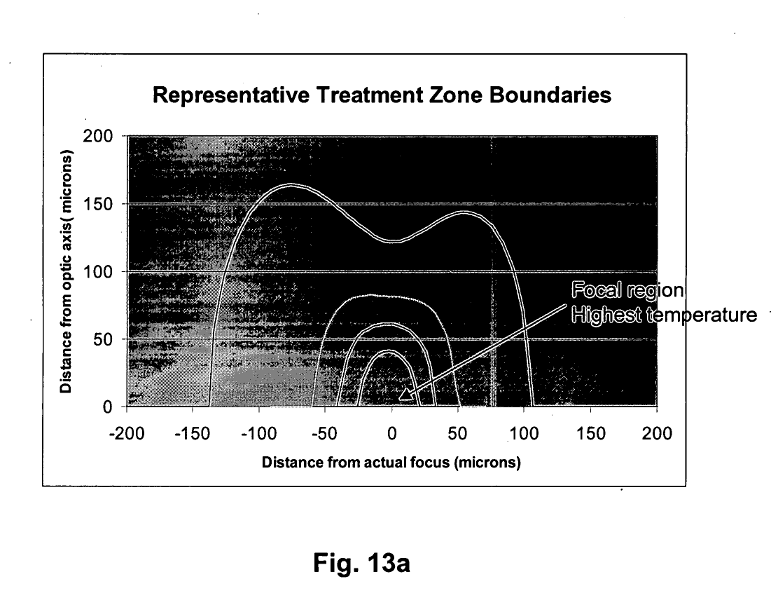

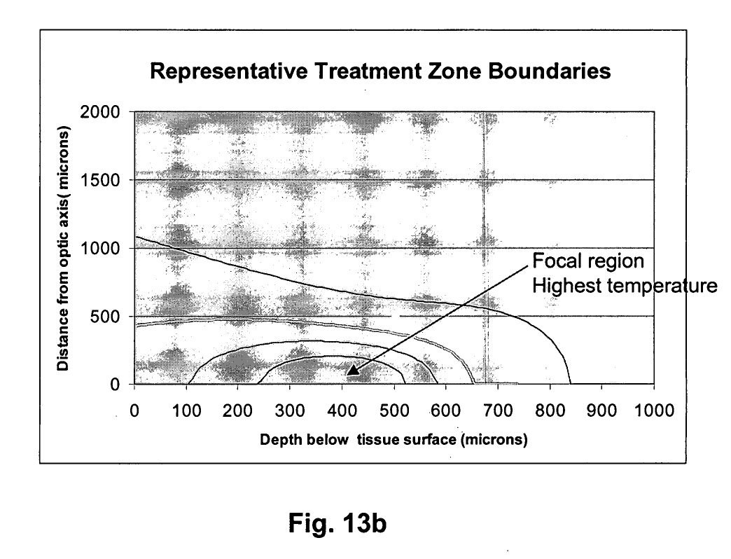

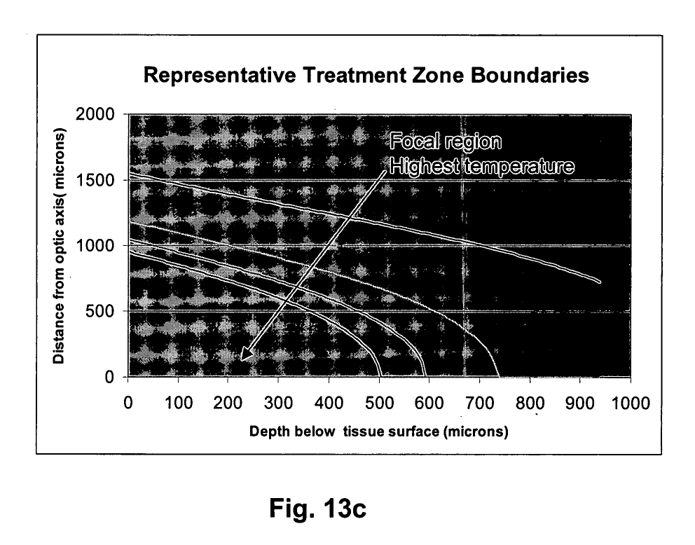

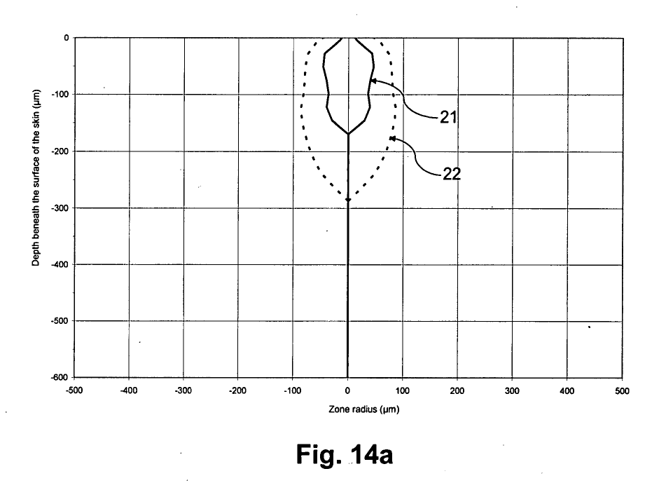

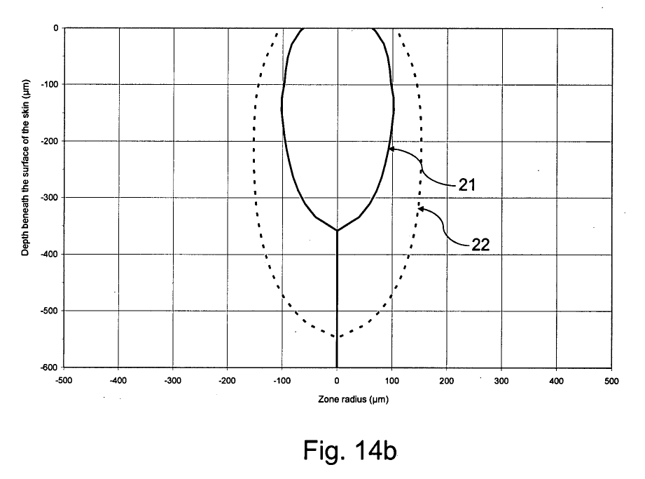

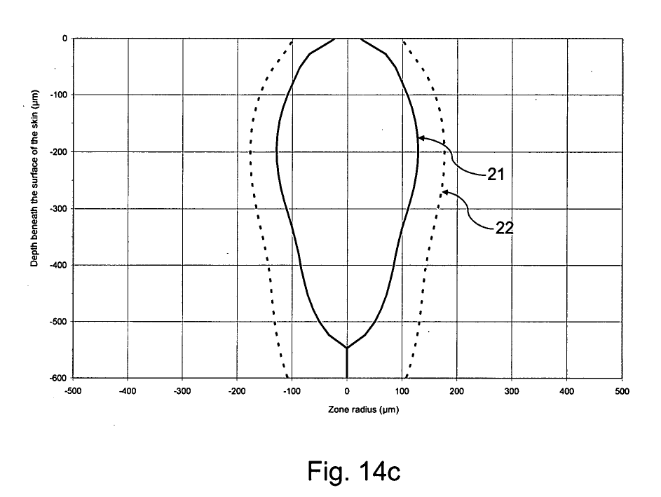

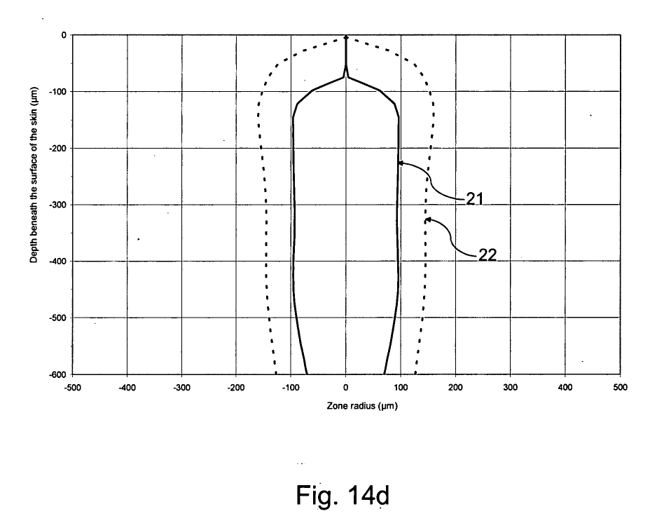

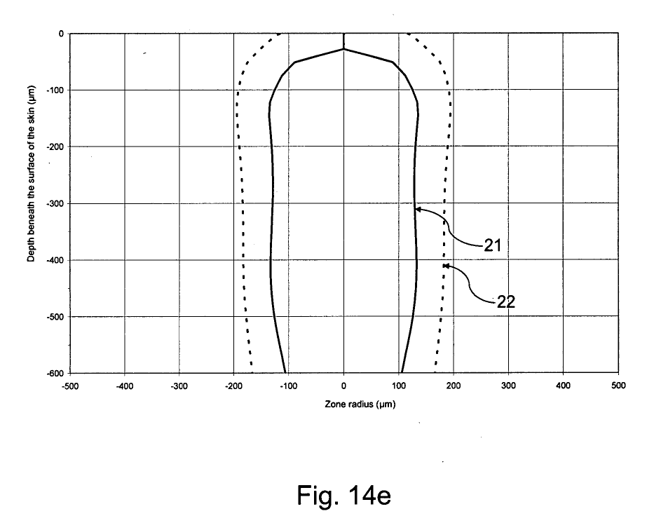

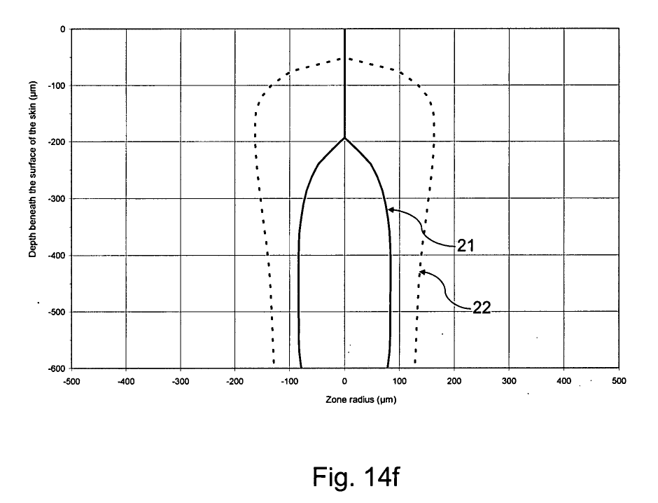

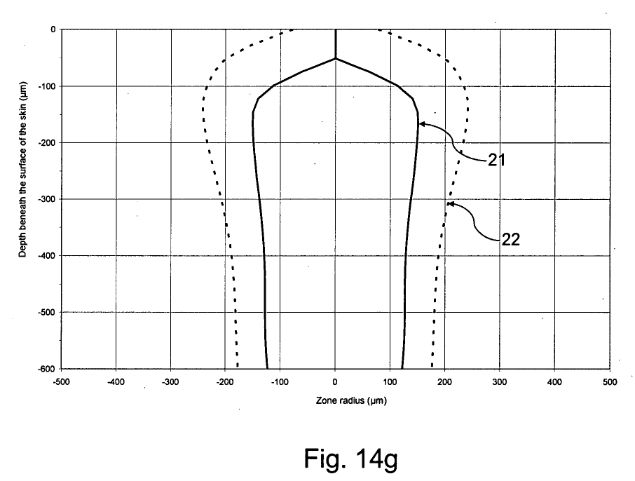

One important aspect of thermal diffusion that is evident in FIGS. 13 and 14 is that the temperature gradients are favorable for heat transport of heat deeper into the tissue than the laser light itself penetrates. Thermal diffusion may add up to 200 microns or more to the depth of a treatment zone as a result of this longitudinal heat transport.

Representative results using the model are presented in FIGS. 13a-13c which illustrate the range of treatment zones achievable by adjusting the focusing strength of the beam incident on the surface. The various contour lines on the graphs indicate contours of constant temperature. These representative results are consistent with typical treatment results using embodiments of the present invention on humans.

For example, FIG. 13a illustrates the type of zone boundaries that are predicted by this model. The parameters were set to the following: μ=100/cm, θ=100 mrad, R=1 mm, w=50 μm, f=500 μm, and α=20/cm. The actual focus is at z₀=495 μm, and the actual beam waist is 81 μm. This corresponds to tight focusing to a point 500 microns below the tissue surface.

In FIG. 13b, the parameters are set to the following: μ=100/cm, θ=100 mrad, R=1 mm, w=500 μm, f=500 μm, and α=20/cm. The actual focus is at z₀=495 μm, and the actual beam waist is 504 μm. This corresponds to weak focusing of the beam to a point 500 microns below the tissue surface.

In FIG. 13c, the parameters are set to the following: μ=100/cm, θ=100 mrad, R=1 mm, w=950 μm, f=500 μm, and α=20/cm. This corresponds to using a collimated incident beam.

FIGS. 14A, 14B, 14C, 14D, 14E, 14F and 14G further illustrate different shapes in the thermally altered tissue (i.e. necrotic zone 21 and HSZ 22) caused by embodiments of the present invention. For example, the treatment parameters that are used to produce the treatment zone in FIG. 14A result in a necrotic zone 21 that has its largest diameter in the epidermis, with a HSZ 22 that is approximately 200 μm in diameter. A different set of treatment parameters is used to produce the necrotic zone in FIG. 14D. These parameters result in a necrotic zone that penetrates significantly deeper into the skin and has a significantly smaller radius within the top 100 μm of the skin. In addition, these parameters result in a HSZ 22 that is significantly wider and deeper than the corresponding HSZ of FIG. 14A. The shape of the treatment zone will dictate to a large extent the shape of the HSZ, as a HSZ is generated in part by thermal diffusion of the heat energy deposited in the necrotic zone. The shape of the necrotic zone can be controlled by the appropriate combination of one or more of the laser beam spot size, fluence (energy per unit area), pulse duration, energy per pulse, laser wavelength, optical beam profile, system optics, lotion, contact tip temperature, surface cooling, and contact tip thermal conductivity.

For example, a circular laser beam of 1500 nm wavelength emitted from a single mode fiber, focused to a depth of 615 μm within the skin with a pulse energy of 12 mJ, an exposure time of 12 ms, a peak power of 1 W, an optical magnification of approximately 6:1 (i.e., the image is 6 times smaller at the focal point in comparison with the object at the output of the fiber when focused in air instead of in skin), and a passively cooled glass plate in contact with the skin through an optically transparent index matching lotion will create a necrotic zone 21 that is approximately cylindrical as shown in FIG. 14D. Cross sections of several such necrotic regions 21 are shown in FIGS. 14A, 14B, 14C, 14D, 14E, 14F and 14G. For this type of treatment, the resulting necrotic zone 21 will be approximately 100 to 300 μm in diameter (perpendicular to the direction of the incident beam) and approximately 150 to 900 μm deep in the direction of the beam. FIGS. 14A, 14B, 14C, 14D, 14E, 14F and 14G further illustrate the shape and depth of the thermally altered zones 22 that may be created by various combinations of laser pulse duration, pulse energy, and focal depth. In these figures, the y axis shows the depth of penetration of the thermally altered zone from the surface of the skin, where 0 is the skin surface and −600 would indicate 600 μm into the skin. The x axis shows the size of the altered tissue zone in the radial direction. FIGS. 14A, 14B, 14C, 14E, 14F and 14G show shapes of the treatment zone 21 and the HSZ 22 that may be generated by using the same parameters as used for FIG. 14D, but with changes in the pulse duration, pulse energy, and focus depth as described in Table 1. As can be seen by examining FIG. 14C, necrotic zones can be created that are non-cylindrical.

TABLE 1

| Pulse Duration | Pulse Energy | Focus Depth Below The |
|---|---|---|
| (msec) | (mJ) | Surface Of The Skin (μm) |
| 3 | 3 | 55 |
| 12 | 12 | 55 |
| 12 | 12 | 335 |
| 12 | 12 | 615 |
| 20 | 20 | 615 |
| 12 | 12 | 755 |
| 25 | 25 | 755 |

Typical aspect ratios for treatments using embodiments of the present invention should typically be greater than about 1:2 (or 1-to-2), and preferably greater than about 1:4. For example, an aspect ratio of 1:2 would mean that for every 1 micron of diameter of the necrotic zone, there is 2 microns of depth of the necrotic zone. Aspect ratio is the cross-sectional width (e.g., diameter for circular cross-sections) of the necrotic zone (i.e. typically at its widest point in a direction perpendicular the optical axis of the treatment beam) divided by the total depth of the necrotic zone measured along the optical axis of treatment of the optical radiation. Cross-sectional width is measured across the largest cross-sectional area of the necrotic zone, and the cross-sectional width is the smallest distance across the cross-sectional area along a line that includes the center of the cross-sectional area. Depth is measured from the top of the necrotic zone to the bottom of the necrotic zone along the optical axis of the optical radiation. For example, FIG. 12h illustrates an example of an elliptical cross-sectional area 1244, and the cross-sectional width is the minor axis 1246. An aspect ratio can be defined similarly to include the diameter and depth of the HSZ.

### EMBODIMENTS AND EXAMPLES

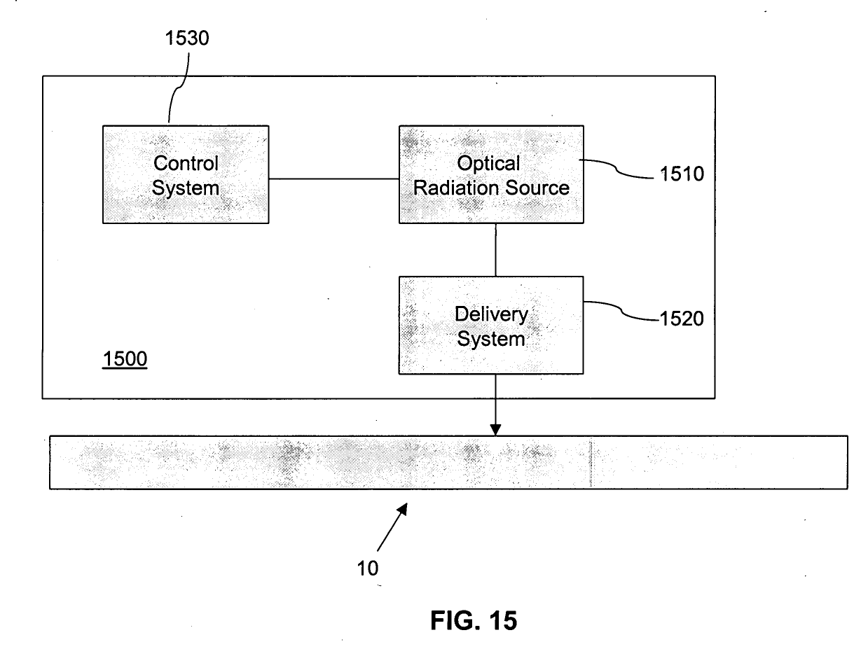

One embodiment of the apparatus used for practicing this invention is shown in FIG. 15. Apparatus 1500 comprises a control system 1530, an optical radiation source 1510, and a delivery system 1520 to deliver the desired pre-determined treatment pattern to the target tissue 10. The control system 1530 is operably connected to the optical radiation source 1510 and the delivery system 1520. The control system 1530 may include separate control systems (not shown) for the optical system and the delivery system. For certain applications, the optical radiation source 1510 includes multiple laser light sources, which can be arranged in an array, such as a one-dimensional array or a two-dimensional array.

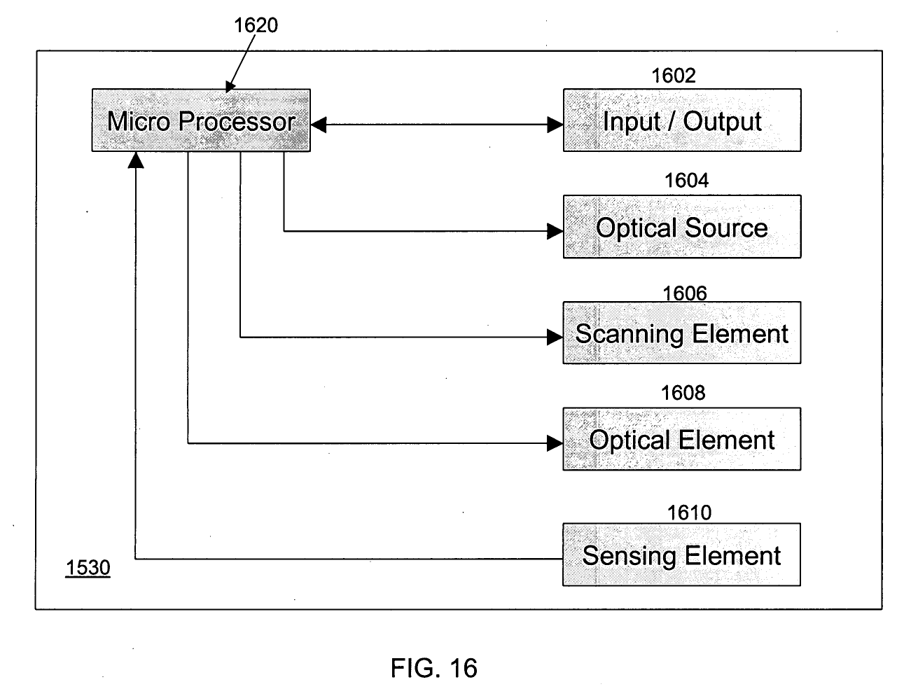

FIG. 16 shows a block diagram of the control system 1530. Control system 1530 is operably connected to the input/output 1602, the optical source 1604, the scanning element 1606, the optical element 1608 and the sensing element 1610. Input/Output 1602 could be a touch screen element or other such means that are well known in the art. The sensing element 1610 may include an optical, mechanical or electrical sensor or detector, such as, for example, an optical mouse, a mechanical mouse, capacitance sensor array or profilometer.

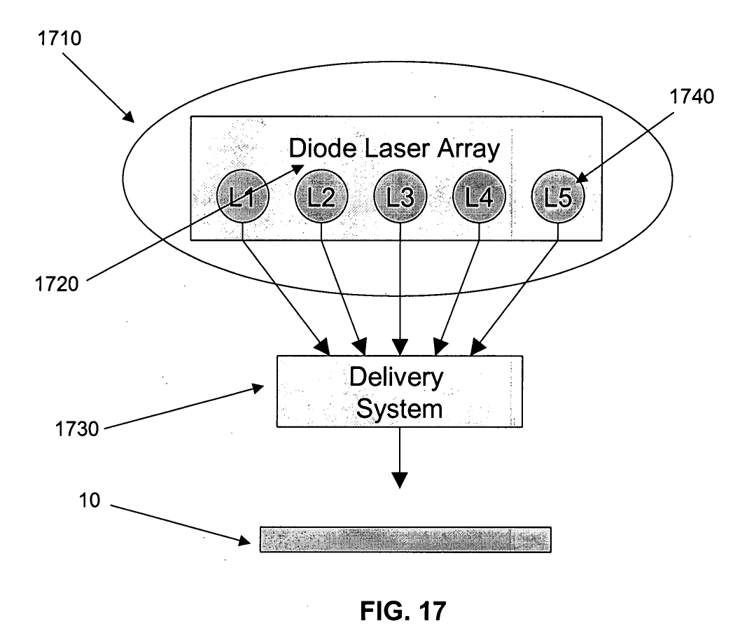

FIG. 17 shows an embodiment in which the optical source 1710 includes laser light sources 1740 arranged in a one-dimensional array 1720. A laser light source can provide one or more optical beams having particular optical parameters, such as optical fluence, power, timing, pulse duration, inter-pulse duration, wavelength(s), and so forth, to produce a desired dermatological effect in the target tissue 10. The wavelength is typically chosen largely based on target chromophore whether naturally found in the skin, such as, for example, water, hemoglobin or melanin, or added to the skin via topical or injection, such as, for example, drugs incorporating or attached to a chromophore. By way of example, a laser light source can provide an optical beam having a wavelength or range of wavelengths between approximately 400 nm and 12,000 nm, such as between approximately 500 nm and 3,000 nm, or preferably between about 1000 nm and about 2000 nm, or more preferably between about 1400 nm and about 1600 nm. For example, for purposes of non-ablative coagulation of a dermal layer of the targeted portion 10, a laser light source can provide an optical beam having a wavelength of approximately 1,500 nm and an optical fluence incident on the outer surface of the skin between approximately 0.001 Joules/cm² and 100,000 Joules/cm², such as between approximately 1 Joules/cm² and 1000 Joules/cm² . The energy would typically be in a range less than about 100 mJ per pulse, with a pulse duration less than about 100 msec. For certain applications, the pulse duration of an optical beam can be approximately equal to or less than a thermal diffusion time constant, which is approximately proportional to the square of the diameter of a focal spot within the targeted portion, associated with the desired treatment zone. Pulse durations that are longer than the thermal diffusion time constant can be less efficient and cause the focal spot to expand or shrink undesirably by thermal diffusion. This is one approach for making HSZs 1004 overlap, as shown in FIG. 10.

Examples of optical radiation sources include, but are not limited to, diode lasers, diode-pumped solid state lasers, Er:YAG lasers, Nd:YAG lasers, Er:glass lasers, argon-ion lasers, He—Ne lasers, carbon dioxide lasers, excimer lasers, fiber lasers, such as erbium fiber lasers, ruby lasers, frequency multiplied lasers, Raman-shifted lasers, optically-pumped semiconductor lasers (OPSL), and so forth. For certain embodiments, a laser light source is desirably a diode laser, such as an infrared diode laser. The optical radiation sources may be continuous wave (CW) or pulsed. However, it should be recognized that the selection of a particular type of laser light source in the optical system is dependent on the types of dermatological conditions to be treated using the dermatological apparatus 1500. In FIG. 17, the optical radiation source 1710 could include one particular type of laser light source capable of providing one wavelength or wavelength range. Alternatively, the optical source 1710 could include two or more different types of laser light sources to provide a variety of different wavelengths or wavelength ranges. Optical beams from different laser light sources can be directed to the targeted portion 10 on a one-by-one basis or at the same time. In addition, one skilled in the art will recognize that while laser sources are the preferred embodiment of the optical source described here, other optical sources such as a flashlamp, an optical parametric oscillator (OPO) or light-emitting diode could also be used.

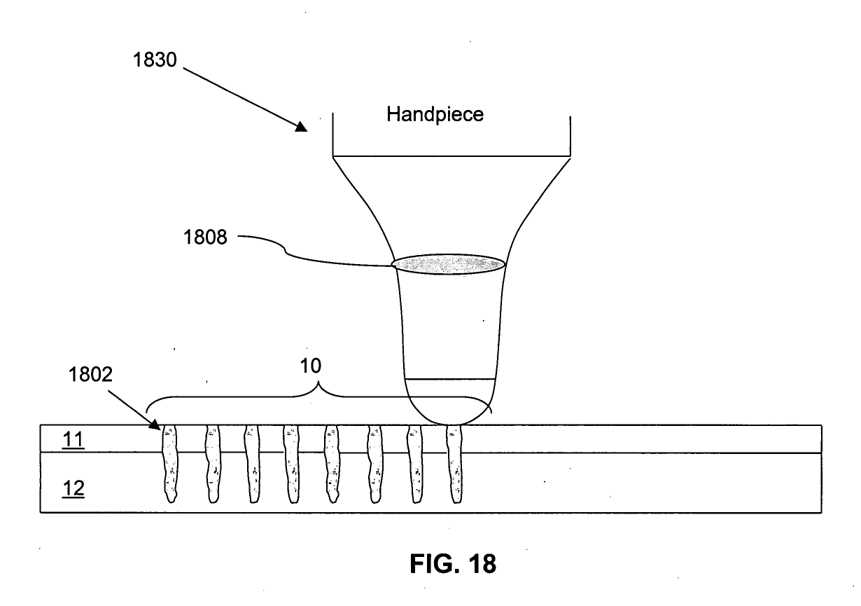

Referring to FIG. 18 as another embodiment, the optical delivery system 1830 also includes an optical element 1808 that is optically coupled to the optical source (not shown). The optical element 1808 has a numerical aperture greater than about 0.005, can be either a collimator or a focusing element and functions to direct optical energy from the optical source to the targeted portion 10. In the present embodiment, the optical element 1808 directs optical energy to the targeted portion 10 by focusing the power of the optical energy to one or more treatment zones 1802 within the target tissue 10. Desirably, multiple treatment zones are simultaneously or sequentially exposed to optical energy. Multiple treatment zones can be separated from one another so as to form discrete treatment zones. Alternatively, or in conjunction, multiple treatment zones can intersect or overlap one another.

In the present embodiment, the optical element 1808, in conjunction with the delivery system, directs optical energy in a pattern, such as a discontinuous or microscopic pattern, so that one or more treatment zones are exposed to optical energy sequentially or simultaneously. Use of a pattern of optical energy provides greater efficacy of treatment by allowing for control of the fraction of the target tissue 10 that is exposed to optical energy. Different patterns can provide a variety of different thermally altered zones and a particular pattern can be selected based on the type of dermatological condition to be treated. For instance, in the case of a sensitive dermatological condition such as dermal melasma or deep pigmented lesions, the use of a pattern of optical energy permits an effective level of treatment within multiple treatment zones. At the same time, by controlling the fraction of the targeted portion 10 that is exposed to optical energy, pain, immune system reaction, trauma, and other complications can be reduced. By having the treatment zones adjacent to healthy and substantially undamaged cells, healing of the targeted portion 10 is quicker, since the possibility of congestion or impairment of repair processes is reduced. Use of a pattern of optical energy also can facilitate multiple treatments to produce a desired effect by allowing safer individual fractional treatments to be combined to produce a significant result. This is typically milder and poses a lower risk to the patient. Furthermore, visible impressions of treatment can be reduced by using a pattern of treatment where an individual treatment zone is on the same or smaller scale than the normal visible texture or constituents of the skin itself. Such reduced visible impressions may mean that the necrotic zones are sub-surface or have surface cross-section dimensions less than about the size of skin pores. Such reduced visible impressions may mean that individual necrotic zones are substantially not visible to the naked human eye observing from 3 feet or more away from the skin surface. Predetermined patterns may be chosen based on the effect desired in the tissue. Such patterns may be uniform or non-uniform, as may the individual treatment zones. Predetermined patterns may include polygonal grids, circular patterns, spiral patterns and others. Such patterns may be formed using one or more optical sources irradiating in a sequential, random pattern or interleaved firing mode. The resulting pattern may alternately be random.

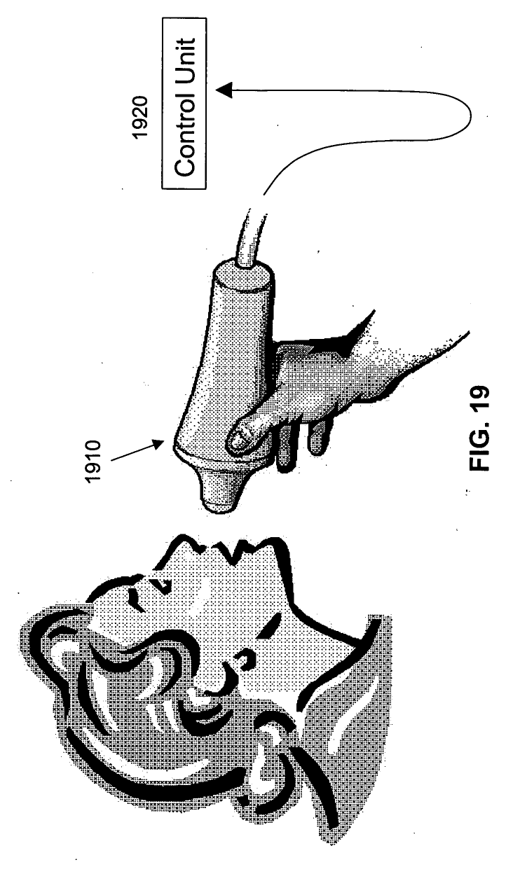

FIG. 19 illustrates another embodiment in which a hand-piece 1910 is sized and configured to be used by an operator in treating a patient's skin according to various embodiments of the present invention. The hand-piece is operably coupled to the control unit 1920.

### Selection of Parameters

In accordance with the inventions disclosed herein, for treatments near the surface of the tissue, there is great latitude in the selection of irradiation parameters, as the heat-affected zones can be limited to a small region by focusing of the light, or by other means such as optical interference.

For deeper treatments, the benefits of the present invention are obtained using any one of a number of combinations of parameters for the irradiation system, as outlined herein based in part on the above model. With respect to the irradiation source, the wavelength may be adjusted to optimize both the tissue absorption and scattering. For example, to achieve treatment zones centered at a depth of 1 mm, the absorption coefficient should be about 10/cm, if scattering is low, and less than this for deeper treatments.

The absorption in human tissue in the visible light range is mostly due to specific chromophores (such as hemoglobin or melanin) and scattering is generally too strong to meet the conditions given herein for deeper treatment zones. In the near-infrared radiation range, water is typically the only, or vastly the most significant, chromophore. The absorption coefficient for water in the near infrared range has peaks near 1450 nm (i.e. absorption coefficient of about 30/cm) and 1950 nm (i.e. absorption coefficient of about 200/cm) and between these peaks it does not drop significantly below 10/cm. Above the peak at 1950 nm the absorption does not drop to small values but increases to extremely high values comparable to the absorption of Er:YAG laser light and/or CO₂ laser light. Between 1000 nm and 1450 nm the absorption coefficient rises steadily, and can be as low as 2/cm or less. Below about 1000 nm, chromophores such as hemoglobin and melanin become more prevalent, and water absorption recedes. Thus, in the wavelength region between 1000 nm and 2000 nm, the absorption of skin is in the range suitable for efficient treatments to depths of a few mm or less. In this wavelength range, the scattering strength (i.e. the scattering constant) of skin is about 100/cm but is peaked forward so that the effective extinction rate by scattering is substantially reduced, and in fact weak enough to allow significant penetration of focused light to a few millimeters depth, without excessive spreading of the light energy. This combination of relatively weak absorption and scattering in this wavelength range is attractive for the formation of columnar treatment zones at depths up to a few mm.

The laser power should be adjusted so that there is just enough optical energy introduced into the skin to create the desired necrotic zones and HSZ zones. An excess of energy will create larger zones than desired, whereas a lack of adequate energy may fail to create the desired zone at all. There is greater latitude in the pulse length of the optical pulse. The pulse length should be chosen long enough to avoid excessive intensity at the skin surface, but short enough to avoid significant heat transport during the pulse. For a zone of dimension L, the pulse length is proportional to L²/D, and optimizes at about L²/4D, where D is the effective diffusion coefficient. This typically amounts to about 1 ms for a zone size of 100 microns. Longer pulse widths will create larger treatment zones and will require greater pulse energy than the minimum required. In this regard, Q-switching may cause undesirable tissue damage, but if high intensity is desirable, then Q-switched laser systems may be used to advantage in obtaining fractional treatments, especially for treatment zones within 100 microns of the skin surface.

Yet another means for controlling the treatment zones is to use more than one light source. Such sources may be directed through the same aperture to the skin, or through separate apertures. They may be applied simultaneously or sequentially, or interleaved in any way. Each source creates its own temperature profile, so that the actual temperature profile is the sum of all the individual profiles. Thus, a band of wavelengths, such as is provided by some diode lasers, will create treatment zones that are elongated columnar zones. Use of two wavelengths may create a treatment zone that is a combination of a deeper and a shallower zone, and so on. Moreover, frequency chirped pulses may also be used in this way. One of ordinary skill will recognize the potential for further fine adjustment of the shape and depth of the treatment zones using multiple sources of different wavelengths or directed through different apertures to the skin surface with appropriate temporal sequencing.

Embodiments of the present invention wherein pulses are interleaved provide treatments where a response of the tissue to one wavelength conditions the tissue for an enhanced response at another. For example, a first treatment beam is applied having a given wavelength, pulse duration, energy and beam diameter calculated to heat the tissue. A second treatment beam is then applied to coagulate the heated tissue starting at the higher base temperature caused by the first treatment beam. Alternately, a first treatment beam may target one chromophore, while the second treatment beam targets a second different chromophore.

It will also be evident to one of ordinary skill that there are many optical means of directing light to the skin surface in order to create a desired pattern of energy at or below the skin surface. These include, but are in no way limited to, lenses, mirrors, beam splitters, fiber optics, diffraction gratings, diffractive elements and holographic elements. Any and all such means are within the scope of the inventions disclosed herein in that they may be used, individually or in combination with each other, to create a pattern of irradiation and thereby control the shape of the treatment zones. In particular, any means of creating a substantially columnar treatment zone is within the scope of this invention.

Another aspect of this invention is the arrangement of the individual treatment zones such that healthy, un-treated tissue is left between zones of heat-affected or treated tissue. Means of creating a pattern of individual treatment zones include, but are not limited to, fly's eye lenses, acousto-optic and electro-optic deflectors, diffractive elements, galvanometers, piezo-electric devices, MEMS, and rotating scanning elements. Scanner technology is well-developed and may be applied to this function. One embodiment employing scanner technology includes a device wherein the scanning function is included in a hand-piece or head which moves slowly over the tissue surface, while applying many optical pulses that each create an individual treatment zone. The separation between the treatment zones is a critical parameter for fractional treatments and is best accomplished using technology that controls the pattern of irradiation sites precisely. However, the motion of optical parts within the head, coupled with the finite pulse width of each individual pulse, causes the optical pulse to sweep, or blur, over a small but finite path during irradiation. Such blurring can be controlled by making the pulse length short, or by slowing the motion of the moving optical components, or by active control of the blurring process (i.e. de-blurring). The first two options have the consequence of limiting the area of the patient's skin that can be covered per unit time. However, de-blurring of the irradiation pattern enables a greater area of skin to be treated per unit time. Accordingly the de-blurring function lies within the scope of our invention to the extent that it keeps the individual treatment zones sharp, yet enables a rapid scan over the patient's skin treatment area. Typically such a rapid scan includes moving a handpiece or a delivery system portion at up to about 10 centimeters per second. An embodiment including such de-blurring is found in co-pending U.S. patent application Ser. No. 10/750,790, filed on Dec. 31, 2003, which is incorporated herein by reference.

### Alternate Embodiments

As will be evident to one of ordinary skill in the art, there are many possible configurations of laser sources, optics and hardware that provide a means of controlling the shape, location and pattern of the treatment zones according to our invention. The following embodiments and examples represent varying degrees of sophistication in implementing means of creating treatment zones in human tissue using the teachings provided herein.

One embodiment of the invention is to utilize a compact diode laser or a fiber laser as a source of optical energy. The source is located conveniently near the patient, and the light energy is transported to the immediate vicinity of the treatment area using optical fibers. In general, the optical energy emerging from the optical fiber has some, but not all of the characteristics of the light that are required by the tissue treatment being performed. The fiber terminates in a hand-piece that is held by the practitioner over the treatment area. The function of the hand piece is to perform a local and final conditioning of the optical energy to have the correct parameters as described herein, so that the desired result is obtained in the tissue. The practitioner applies one or more optical pulses to the treatment zone, moves the hand-piece to another area to be treated and repeats the application.

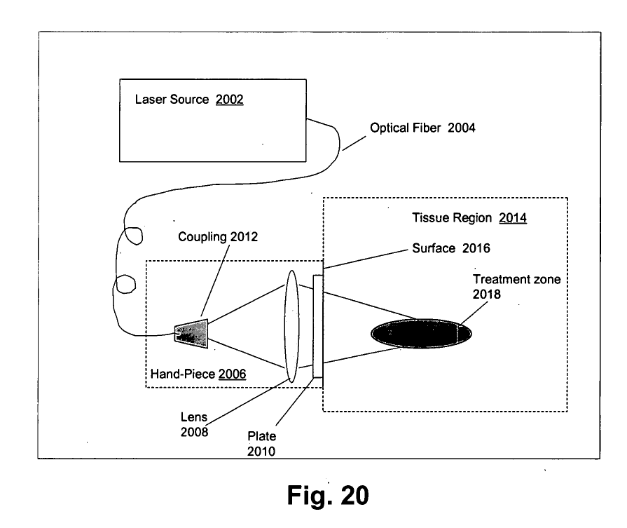

For example, the light source may be a diode or fiber laser operating at 1550 nm. As illustrated in FIG. 20, the laser 2002 is coupled into a fiber 2004 which terminates in a hand-piece 2006 that contains a lens 2008 or combination of lenses and a flat optical plate 2010 which is placed by the practitioner in close contact with the tissue surface 2016. The light emerges from the fiber, passes through the lens and then through the plate. The diode laser is set to deliver a pulse of light of precisely controlled power and pulse length. The lens collimates the light and the plate provides a small stand-off between the lens and the tissue, so that the lens is always the same distance from the tissue surface. In this way, a precisely controlled application of light creates a treatment zone 2018. Many variations of this basic design will be immediately apparent to one of ordinary skill in the art, and are embodiments of this invention. These include replacing the lens by a lens combination, as might be utilized to obtain high numerical apertures up to NA=1.0 or even higher (if there is no air gap), and making the plate very thin. This high numerical aperture configuration may be used to create columnar zones in the manner described herein. Further, the plate may be omitted so that the lens or lens combination is in direct contact with the skin. Mirrors, holographic elements and phase plates are some of the equivalent means of creating the degree and extent of focusing required to obtain the desired tissue treatment. The laser pulses are typically released into the fiber at time intervals controlled by the practitioner, through a button or equivalent on the hand-piece, or by a foot pedal (not illustrated). Alternately, a continuous wave (CW) laser beam is released into the fiber and a control mechanism is coupled to the output end of the fiber so that practitioner control is exercised at the fiber end just prior to the beam exiting the system. This embodiment “stamps” the laser pulses onto the tissue, one pulse and one zone at a time. The pattern of treatment zones is determined by the practitioner as he/she relocates the hand-piece between pulses. Alternately, the hand-piece may be in motion with intermittent firing of the laser either based on user control or by an automated system, with a constant repetition rate for firing the laser or a rate of repetition based on the movement of the hand-piece.

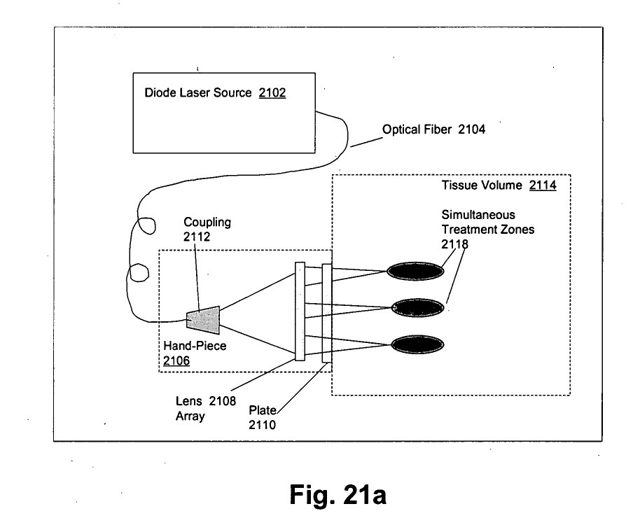

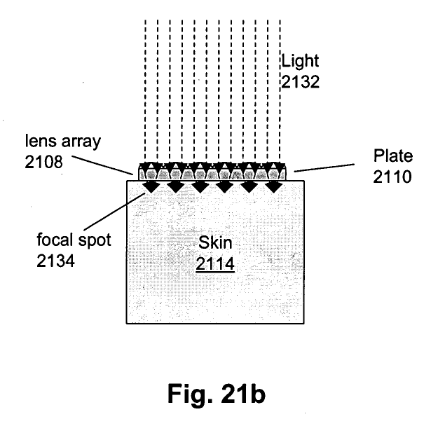

Another embodiment illustrated in FIGS. 21a and 21b utilizes the simultaneous stamping of many pulses through the use of a lens array. The light from the fiber 2104 passes through a close-packed array of lenses 2108 to create a number of treatment zones 2118 simultaneously. One advantage of the lens array is that it defines precisely the location of many treatment zones, and so fixes precisely the fraction of the tissue that is treated. Lens arrays may be fabricated as a simple array of normally refractive lenses cut or etched into a single transparent plate. Greater optical efficiency may be obtained using a diffractive optic such as a phase plate or zone plate in the manner of a Fresnel lens. Holographic approaches are also known. A lens array is just one of many means of realizing the embodiment of simultaneous stamping of many pulses. All such means are within the scope of the invention.

A further lens array embodiment includes the use of a silicon lens array to convert a single beam to an array of small treatment zones simultaneously within the skin such that rapid treatment can occur. As illustrated in FIG. 21b, these lenses can be placed in contact with the skin directly or through a contact window or plate to create a very high NA system if small treatment zones or high angles are desired, as in the case of deep dermal treatments. A second aspect of this embodiment is that a micro lens array can be built into an adapter tip that can be used to convert an existing medical laser device into a device with small treatment zones (<1 mm diameter). Microlens arrays are commonly created using etching or molding materials such as glass or silicon. For example, companies such as MEMS Optical (Huntsville, Ala.) make etched silicon lens arrays and Corning (Corning, N.Y.) and Lightpath Technologies, Inc. (Orlando, Fla.) molded glass lens arrays. Other materials such as UV cured epoxy manufactured by Oriel Instruments division, Stratford, Conn. of Spectra Physics, Inc., Mountain View, Calif., may be used. Diffractive elements such as those manufactured by Holographix, Inc. Hudson, Mass., may also be used to form microlensing elements. In addition, an array of small GRIN lenses, such as may be manufactured by Dicon Fiber Optics, Inc., Richmond, Calif., or other small lenses (Lightpath Technologies, Inc. Orlando, Fla.) could be joined together to create an array.

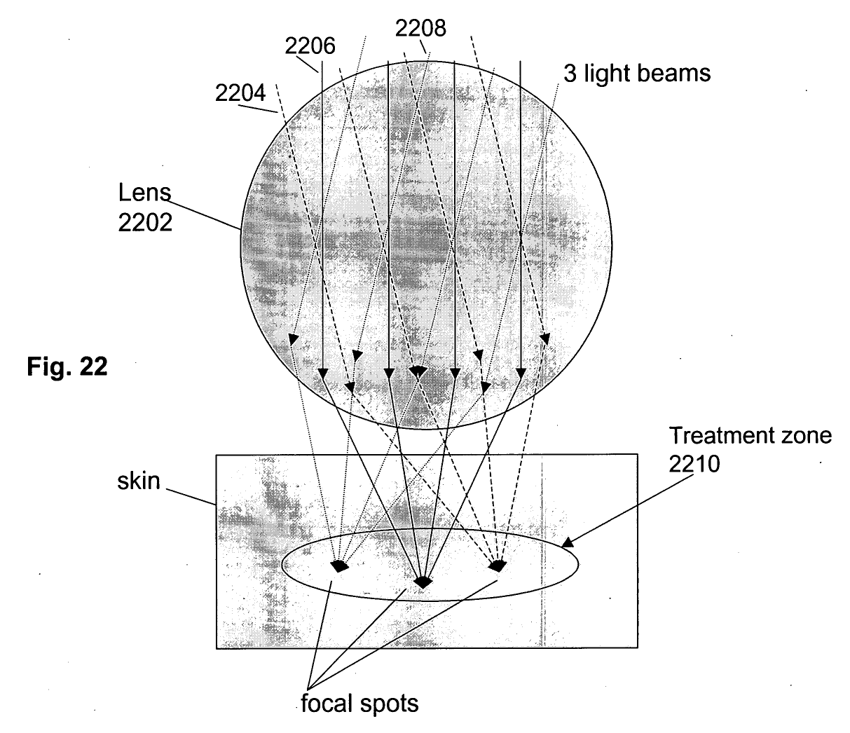

For certain applications of microscopic laser treatment, it is desirable to have a large area at the surface of the target area and a small area at the focal point of the laser system. This can be achieved by employing embodiments of the present invention that have a high numerical aperture lens system. If multiple spots are desired, and a conventional multiple separate adjacent lens system is used, there is a limit on how closely multiple lens elements may be packed together. Two filled individual lenses cannot be placed any closer than edge to edge without having their beams overlap. For a particular lens array with normal incidence relative to the target skin, this places a limit on how closely together their focal spots can be placed. As illustrated in FIG. 22, an embodiment of the present invention includes using a single large lens to create multiple spots within the skin in close proximity. This embodiment describes a design for creating multiple spots very close together using a single lens instead of a lens array. Multiple light beams (2204, 2206, 2208) are incident at different angles on a single large lens 2202 that focuses those beams to different places within the skin to create a treatment zone 2210. Multiple light beams can be incident on a spherical lens to create multiple spots within the skin. The beams come to different focal spots because they are incident on the lens at different angles. Other lens shapes and optical configurations will be evident to one skilled in the art, and these other lens shapes and optical configurations are alternate embodiments of the present invention.

A further embodiment of the invention uses a diode laser mounted together with the lens in the hand piece. The light from the diode lasers is directed to the tissue directly by a system of lenses and/or mirrors that may either reshape the beams or focus them, or both. Electrical and thermal conditioning of the diodes is typically more complex because the main power supply and a substantial part of the cooling mechanism may be placed remotely. Alternately, the power supply and cooling mechanism may be placed within the handpiece.

A further embodiment is a variation on the lens array design, and includes directing the laser beam from a single laser sequentially from one lens to the next, or one irradiation site to the next, by a scanning device. Thus, the power of the laser is directed for a short time to each lens or to each site, in contrast to the case of simultaneous illumination of all the lenses, where the laser power is divided between the lenses and sites. For a fixed laser power and treatment energy per site, the total time the laser is emitting optical energy is the same in the sequential and simultaneous cases. However, the time of irradiation of any one site is much shorter for sequential illumination than for simultaneous illumination. A short pulse length is often advantageous for controlling the shape of the treatment zones. While many effects in tissue depend on the total energy delivered, or the peak temperature reached, there are other effects that depend on the rate of heating. For example, the electrical response of nociceptor cells lies in this latter category. Thus, the pulse length may significantly influence the experience of pain by the patient. We have already described the role that pulse length may have in expanding the diameter of columnar zones. If the pulse length is limited by this (or another) consideration then sequential illumination is a means of reducing the power of the optical source and thereby reducing the cost and the size (footprint) of the irradiation hardware.

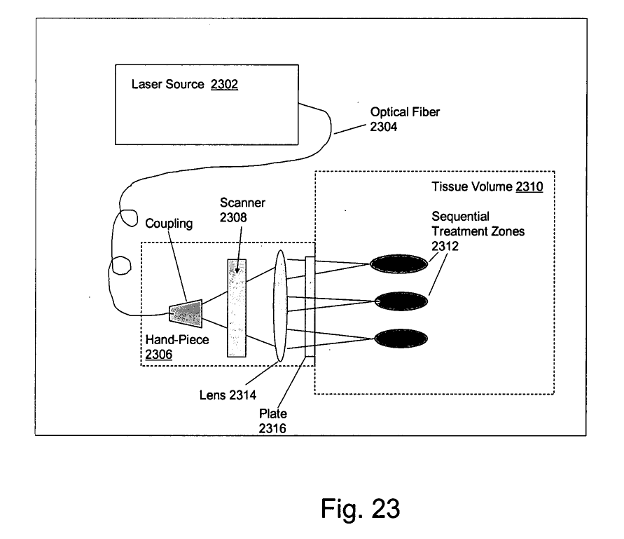

As illustrated in FIG. 23, a further embodiment is to locate the laser remotely, and sequentially scan the beam(s) using a scanner 2308 and a single lens 2314. The scanner may reside between the lens and the tissue 2310, or it may reside between the lens and the output of the optical fiber 2304. The scanner 2308 directs the optical energy to different sites in a predetermined sequence. The scanner may utilize any suitable method of redirecting a laser beam, such as acousto-optic deflectors, MEMS devices, galvo-activated mirrors, or rotating mirrors. In one embodiment, a pair of galvo-driven mirrors redirects the laser beam after it emerges from the fiber, and before it passes through a lens that creates a sharp focus below the surface of the skin. The parameters of the scanner, such as its location, angular variation or beam-center motion, may be determined by well-known optics formulae and are well-understood by those skilled in the art. Scanners have the advantage over static systems in that they may be designed to correct for blurring of the treatment zone along the direction of motion of the hand-piece as the hand-piece moves over the skin surface. The parameters describing the motion of the hand-piece may be obtained using a sensor and optical mouse technology. In particular a scanner may be configured to correct real-time for the specific motion caused as the practitioner moves the hand-piece over the tissue surface. The scanner 2308 may be one-dimensional or two-dimensional. The scanner may also be in the third-dimension along an axis parallel to the optical axis so as to create a scanning of the depth of focus of the system.

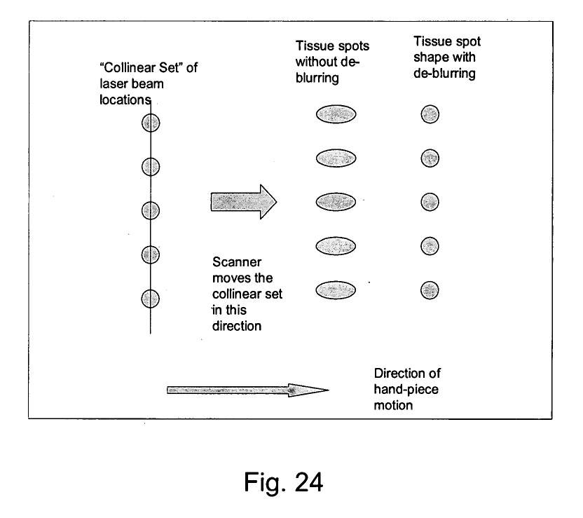

Further embodiments may also be envisaged by one of ordinary skill according to the conventions of the field, and the teachings presented here. For example, the use of several lasers, pulsing together or in sequence, allows parallelism in the treatment of many sites. It also allows some variation in the wavelength used in the treatment protocols. For example, using several different wavelengths enables the treatment zone to be elongated. As illustrated in FIG. 24, if several lasers are used, the sites they are directed to can be arranged to lie along a line perpendicular to the direction of motion of the hand-piece over the tissue. The sites in this ‘collinear set’ are illuminated substantially simultaneously. If the ‘collinear set’ concept is combined with a scanner that moves the entire set of sites, as a group, in the direction of motion of the hand-piece over the skin, such a scanner can be designed to correct for blurring as well. This combination of a collinear set fixed in relation to each other, but scanned as a group in a direction perpendicular to the mathematical line joining them has several attractive features, including reducing the mechanical accelerations in the scanner while de-blurring the laser spots. The collinear set may also be illuminated non-sequentially, randomly or in an interleaved manner to allow for heat dissipation between adjacent treatment sites between treatments of those adjacent sites.

A further alternate embodiment of the present invention includes counter-rotating elements or wheels with optical elements on the counter-rotating elements such that one or more beams passing through the optical elements are deflected and/or focused in a desired direction. Examples of such systems are described in co-pending U.S. patent application Ser. No. 10/750,790, filed on Dec. 31, 2003, and Ser. No. 10/751,041, filed on Dec. 23, 2003, both of which are incorporated herein by reference.

### Experimental Results and Histology

The following table (Table 2) shows examples of average results for various system parameters for embodiments of the present invention.

TABLE 2

| | | Focus in air | Average | Average |
|---|---|---|---|---|
| | | (from contact | Treatment | Treatment |
| Wavelength | Pulse Energy | window) | Depth | Diameter |
| (nm) | (mJ per pulse) | (mm) | (microns) | (microns) |
| 1535 | 10 | 0.3 | 375 | 90 |
| 1550 | 11 | 0.3 | 610 | 85 |
| 1535 | 12 | 0.3 | 380 | 98 |
| 1550 | 13.5 | 0.3 | 600 | 95 |
| 1535 | 20 | 0.3 | 575 | 125 |
| 1550 | 22.5 | 0.3 | 700 | 125 |

The depths and diameters are for the necrotic zones and are averages. This data is offered by way of example only and the present invention is not limited to these values. The speed of treatment may be as much as 10 cm per second, and preferably in a range between about 2 cm/second and 6 cm/second. The stratum corneum may be spared using this embodiment and these parameters, or it can be damaged and/or removed, especially if the contact window is removed and/or the wavelength is changed. Additionally, treatment depths achieved may be as much as 100-200 microns deeper than shown as averages in the Table 2 above. Alternate embodiments listed above may produce similar results for depth, width and aspect ratio. However, each embodiment will have differing treatment speeds, pattern densities, precision, ease of use and efficacy.

Typical system parameters across embodiments include: wavelengths in a range between about 500 nm and about 4,000 nm, and preferably between about 1,000 nm and about 3,000 nm, and more preferably between about 1400 nm and about 1600 nm; pulse energies in a range up to about 150 mJ per pulse, and preferably up to about 50 mJ per pulse; an optical treatment beam cross-sectional width at the tissue surface in a range less than about 500 microns, and preferably in a range less than about 200 microns; a numerical aperture for the system in a range between about 0.005 and about 2.0, and preferably in a range between about 0.01 and about 1.0; a focal depth measured from the tissue surface in a range between about 500 microns above the tissue surface and about 2 mm below the tissue surface, and preferably in a range between about 200 microns below the tissue surface and about 1500 microns below the surface; a pulse duration in a range between about 50 microseconds and about 100 milliseconds, and preferably in a range between about 400 microseconds and about 10 milliseconds; for embodiments that include scanning means, a speed of movement of the hand-piece or the optical beams across the surface of the tissue in a range less than about 10 cm per second, and preferably in a range between about 2 cm per second and about 6 cm per second; and a speed of treatment zone (i.e. necrotic zone and/or HSZ) formation of at least about 100 treatment zones per second, preferably in a range between about 500 treatment zones per second and about 2000 treatment zones per second, and more preferably in a range between about 1000 treatment zones per second and about 1500 treatment zones per second. In scanner systems, the speed of movement of the hand-piece may not be correlated directly with hand movement, especially in embodiments with intelligent robotics using mouse control. The typical results for embodiments employing these parameters typically include the following: depth of treatment up to about 4 mm below the surface; a treatment zone diameter of less than about 1 mm, and preferably less than about 500 microns; an aspect ratio of at least 1:2, and preferably an aspect ratio of at least about 1:4; a treatment zone density in a range up to about 2500 treatment zones per square centimeter per pass of the device across the tissue, and preferably in a range up to about 1000 treatment zones per pass of the device across the tissue; and a separation between the centers of adjacent treatment zones of at least 50 microns, and preferably at least about 150 microns.

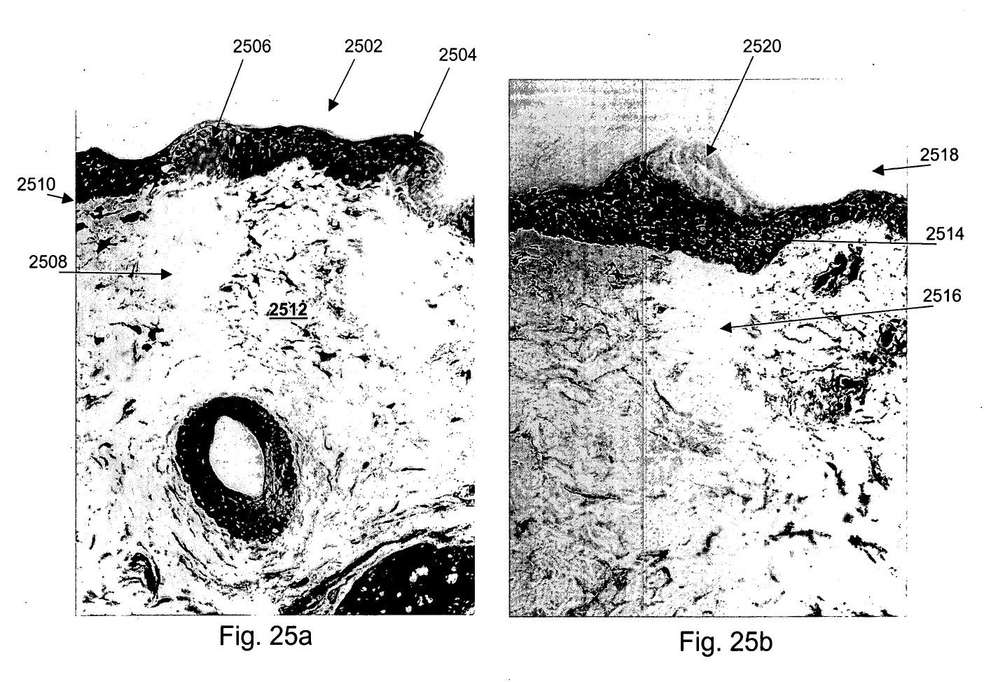

As illustrated in FIGS. 25a and 25b, embodiments of the present invention have been used on human tissue to produce substantially columnar treatment zones that span the epidermal-dermal junction 2510 and spare the stratum comeum 2502. Different system parameters would not spare the stratum comeum, and such sparing of the stratum comeum is not required for all embodiments or treatments. The following parameters were used in treating the tissue shown in FIGS. 25a and 25b: wavelength of 1500 nm and a pulse energy of 5 mJ. FIG. 25a shows the results within one hour after treatment. The stratum comeum 2502 remains intact, the epidermis 2504 is fully coagulated and necrosed, and a substantially columnar thermal wound 2508 is seen in the dermis 2512. A separation in the dermal-epidermal junction 2510 is sometimes seen here as well. The width of the treatment zone is largely uniform throughout the depth of the treatment zone and measures about 80-100 microns. The depth of the wound is about 200-300 microns. FIG. 25b shows the results of the treatment and the healing response 24 hours post-treatment. In FIG. 25b, the epidermis 2504 is largely re-epethelialized in the treated area 2514, dermal repair is continuing in and around the thermal wound area 2516, and often a microscopic epidermal necrotic debris (or MEND) (not shown) has formed under the stratum comeum. The MEND consists typically of necrotic debris from treatment and epidermal pigment. The MEND typically flakes off in less than a week.

The foregoing describes a system and method for laser surgery wherein a focused optical signal such as a laser, LED, or an incoherent source of optical energy is advantageously created to achieve microscopic treatment zones. Further, the foregoing describes a method and apparatus wherein a focused optical signal can be used to treat sub-epidermal regions without damaging epidermal regions. Persons of ordinary skill in the art may modify the particular embodiments described herein without undue experimentation or without departing from the spirit or scope of the present invention. All such departures or deviations should be construed to be within the scope of the following claims.

1. A method for achieving beneficial effects in a target tissue in skin comprising treating the target tissue using optical radiation to create a plurality of microscopic treatment zones in a predetermined treatment pattern, wherein a subset of said plurality of discrete microscopic treatment zones includes individual discrete microscopic treatment zones comprising necrotic tissue volumes having an aspect ratio of at least about 1:2.

2. The method of claim 1, wherein the microscopic treatment zones are separated by thermally unaltered tissue.

3. The method of claim 1, wherein the microscopic treatment zones are surrounded by thermally altered heat shock zones comprising viable tissue.

4. The method of claim 3, wherein the heat shock zones are separated by thermally unaltered tissue.

5. The method of claim 1, wherein the microscopic treatment zones extend from the skin surface up to 4 mm into the tissue.

6. The method of claim 1, wherein the microscopic treatment zones extend from the skin surface to the epidermal-dermal junction.

7. The method of claim 7, wherein the microscopic treatment zones have a depth measured from the epidermal-dermal junction of the skin in a range up to about 4 mm into the dermis.

8. The method of claim 1, wherein the necrotic tissue volumes have a cross-sectional width in a range between about 10 μm and about 1,000 μm.

9. The method of claim 8, wherein the necrotic tissue volumes have a cross-sectional width in a range between about 25 μm and about 750 μm.

10. The method of claim 9, wherein the necrotic tissue volumes have a cross-sectional width in a range between about 50 μm and about 500 μm.

11. The method of claim 2, wherein the cross-sectional width of the viable heat shock zone is controlled by the predetermined treatment pattern.

12. The method of claim 1, where the predetermined treatment pattern of creating the microscopic treatment zones is accomplished by choosing one or more variables from the list comprising laser wavelength, chromophore, laser energy density, pulse energy, pulse duration, thermal diffusion constants and the temporal and spatial distribution of the laser energy.

13. The method of claim 12, wherein the chromophore is water.

14. The method of claim 12, wherein the pulse energy is less than about 150 mJ and the pulse duration is in a range between about 50 microseconds and about 100 milliseconds.

15. The method of claim 12, wherein the pulse energy is less than about 50 mJ and the pulse duration is in a range between about 400 microseconds and about 10 milliseconds.

16. The method of claim 1, wherein the ratio of the sum of the volumes of the microscopic treatment zones to the target tissue volume is less than one.

17. The method of claim 1, wherein the microscopic treatment zones have a physically intact stratum comeum.

18. The method of claim 1, wherein the necrotic tissue volumes are substantially columnar.

19. The method of claim 1, wherein the aspect ratio is greater than about 1:4.

20. A method for achieving beneficial effects in skin tissue comprising treating the tissue by exposing a targeted part of the tissue to optical radiation to create a plurality of microscopic treatment zones such that the volume of the target tissue that remains substantially unaffected by the optical radiation is controlled.

21. The method of claim 20 wherein the control is achieved by focusing the optical radiation to desired depths in the skin.

22. The method of claim 20 wherein each microscopic treatment zone is thermally altered by the optical exposure.

23. The method of claim 20 wherein each microscopic treatment zone is surrounded by a heat shock zone comprising viable tissue.

24. The method of claim 20 wherein the microscopic treatment zone includes a necrotic tissue volume defined by a cross-sectional width in a range between about 10 μm and about 1,000 μm and a depth of up to about 4 mm in the direction of the optical radiation.

25. The method of claim 20 comprising choosing a target region, using a hand piece to deliver laser energy to the target region, where the target region is treated by the movement of the hand piece over the target region when the area of the target region is greater than the cross sectional area of the hand piece.

26. The method of claim 20, wherein a subset of said plurality of discrete microscopic treatment zones includes individual discrete microscopic treatment zones comprising necrotic tissue volumes having an aspect ratio of at least about 1:2.

27. A system for providing dermatological treatment comprising:
a source of optical radiation;
a means for delivering the optical radiation to a target volume of skin;
a control system that is operably connected to the source of optical radiation and the means for delivering the optical radiation;
the control system programmed to control the delivery of optical radiation to the target volume to create one or more microscopic treatment zones such that the volume of the target tissue that remains substantially unaffected by the optical radiation is controlled.

28. A system for providing dermatological treatment, comprising:
a source of optical radiation;
a delivery system operably coupled to the source, the delivery system configured to direct said optical radiation to a volume of tissue in a predetermined pattern; and
wherein the predetermined pattern comprises a plurality of discrete microscopic treatment zones, wherein a subset of said plurality of discrete microscopic treatment zones includes individual discrete microscopic treatment zones comprising necrotic tissue volumes having an aspect ratio of at least about 1:2.

29. The system of claim 28, further comprising a source control system operably coupled to the source, the source control system configured to control a parameter of the optical radiation, wherein the parameter of the optical radiation includes at least one of wavelength, pulse duration, pulse energy, pulse shape, beam profile, chirp and repetition rate.

30. The system of claim 28, wherein the optical radiation has a beam cross-sectional width at the tissue surface of less than about 200 microns.

31. The system of claim 28, further comprising a delivery system controller operably coupled to the delivery system, the delivery system controller configured to control at least one of a plurality of delivery system parameters, the plurality of delivery system parameters including numerical aperture, focal length and optical radiation beam direction.

32. The system of claim 31, wherein the plurality of delivery system parameters further comprise scan speed, scan direction, de-blurring, number of optical radiation beams emitted simultaneously and pattern shape.

33. The system of claim 28, further comprising a contact window located between the delivery system and the tissue, and configured to contact the tissue when the system is in operation.

34. The system of claim 33, wherein the contact window comprises a material that is substantially transparent to the optical radiation and that has a high thermal conductivity.

35. The system of claim 33, wherein the source of optical radiation, the delivery system and the contact window are configured to cause a necrotic volume in an epidermal region within the tissue while substantially sparing a stratum comeum region adjacent to the epidermal region.

36. The system of claim 28, wherein the delivery system further comprises an optical system which includes at least one of a mirror, a lens, a lens array, a diffractive element, a holographic element and a fiber optic element.

37. The system of claim 36, wherein the optical system has a numerical aperture greater than about 0.005 and a focal point located in a range between about 500 microns above the tissue surface and about 1500 microns below the tissue surface.

38. The system of claim 28, wherein the delivery system further comprises a scanner system which includes at least one of a one-dimensional scanner and a two-dimensional scanner.

39. The system of claim 38, wherein the scanner system includes at least one of an acousto-optic element, a piezoelectric element, a galvanometer, a micro-electro-mechanical system (MEMS), a rotating mirror, a rotating prism, an optical mouse and a mechanical mouse.

40. The system of claim 28, wherein the necrotic tissue volumes have a diameter at the tissue surface of less than about 200 microns.

41. The system of claim 28, wherein the necrotic tissue volumes have a depth of at least about 200 microns.

42. The system of claim 28, wherein the discrete microscopic treatment zones have a physically intact stratum comeum.

43. The system of claim 28, wherein the discrete microscopic treatment zones are substantially columnar.

44. The system of claim 28, wherein the centers of the necrotic zones for the discrete microscopic treatment zones are separated by at least 50 microns.

45. The system of claim 28, wherein the predetermined pattern includes uniformly spacing the discrete microscopic treatment zones.

46. The system of claim 28, wherein the predetermined pattern includes a total number of discrete microscopic treatment zones in a range up to about 2500 per square centimeter.

47. The system of claim 28, wherein the optical radiation has a wavelength in a range between about 400 nm and about 12,000 nm, an energy up to about 150 mJ per pulse and a pulse duration up to about 100 milliseconds.

48. The system of claim 28, wherein the optical radiation has a wavelength in a range between about 900 nm and about 3,000 nm, an energy up to about 50 mJ per pulse and a pulse duration in a range between about 400 microseconds and about 10 milliseconds.

49. The system of claim 28, wherein the individual discrete microscopic treatment zones include heat shock zones, the heat shock zones and the necrotic tissue volume for the individual discrete microscopic treatment zones form a substantially cylindrical combined volume, the substantially cylindrical combined volume has an aspect ratio of at least about 1:1.

50. The system of claim 28, wherein the ratio of the sum of the surface areas of necrotic tissue and heat shock zone to the sum of the surface area of untreated tissue within the target tissue volume is less than one.

51. The system of claim 28, wherein the source of optical radiation comprises one or more of a fiber laser, a diode laser, a carbon-dioxide laser, a diode-pumped solid state laser, a ruby laser, and optical parametric oscillator or an excimer laser.

52. The system of claim 28, wherein the system causes an optical fluence incident on the surface of the tissue in a range between about 0.001 Joules per square centimeter and about 100,000 Joules per square centimeter.

53. The system of claim 28, wherein the aspect ratio is greater than about 1:4.

54. The system of claim 28, wherein the delivery system further comprises a handpiece, the system configured to produce up to 2500 necrotic tissue volumes per square centimeter while the handpiece is moving at a speed in a range between about 1 centimeter per second and about 6 centimeters per second.
